(19) United States
(12) Patent Application Publication
(10) Pub. No.: US 2026/0114795 A1
(43) Pub. Date: Apr. 30, 2026

(54) AUTOMATED PLANNING OF COORDINATES AND TRAJECTORIES OF STEREOELECTROENCEPHALOGRAPHY ELECTRODES ON BRAIN TISSUES BASED ON MULTIMODAL DATA USING MACHINE LEARNING

(71) Applicant: University of Pittsburgh - Of the Commonwealth System of Higher Education, Pittsburgh, PA (US)

(72) Inventors: Arka Mallela, Pittsburgh, PA (US); Jorge A. Gonzalez-Martinez, Pittsburgh, PA (US); Thandar Aung, Mars, PA (US)

(73) Assignee: University of Pittsburgh - Of the Commonwealth System of Higher Education, Pittsburgh, PA (US)

(21) Appl. No.: 19/370,470

(22) Filed: Oct. 27, 2025

Related U.S. Application Data
(60) Provisional application No. 63/712,993, filed on Oct. 28, 2024.

(51) Int. Cl. A61B 5/00; A61B 5/37; G16H 50/30

(52) U.S. Cl. CPC A61B 5/4094; A61B 5/37; A61B 5/742; G16H 50/30

(57) ABSTRACT

Presented herein are systems and methods of identifying locations to place stereo-electroencephalography (SEEG) electrodes on brain tissues of subjects for detection of epileptogenic zones (EZs). A computing system may receive, for a subject at risk of or diagnosed with epilepsy, a dataset comprising a neuroimage of the brain of the subject and a metric indicating a degree of neuropsychological function of the subject. The computing system may apply the dataset to a machine learning (ML) model. The computing system may generate a plurality of coordinates for a corresponding plurality of locations along a brain tissue of the subject, each of the plurality of coordinates identifying a plurality of locations at which to place a plurality of SEEG electrodes on the brain tissue to detect an EZ within the subject.

### CROSS REFERENCE TO RELATED APPLICATION

The present application claims the benefit of priority under 35 U.S.C. § 119 (e) to U.S. Provisional Patent Application No. 63/712,993, filed Oct. 28, 2024, which is incorporated by reference in its entirety.

### GOVERNMENT SUPPORT STATEMENT

This invention was made with government support under grant number NS122927 and DC020644, awarded by the National Institutes of Health. The government has certain rights in the invention.

### BACKGROUND

A computing system may process data using a function to generate an output.

### SUMMARY

Aspects of the present disclosure are directed to systems and methods of identifying locations to place stereo-electroencephalography (SEEG) electrodes on brain tissues of subjects for detection of epileptogenic zones (EZs). One or more processors may receive a dataset for a subject at risk of or diagnosed with epilepsy. The dataset may include a neuroimage of a brain of the subject. The one or more processors may apply the neuroimage and the metric of the dataset to a machine learning (ML) model. The one or more processors may generate, based on applying the dataset to the ML model, a plurality of coordinates for a corresponding plurality of locations along a brain tissue of the subject. Each of the plurality of coordinates may identify a corresponding location of the plurality of locations at which to place an SEEG electrode of a plurality of SEEG electrodes on the brain tissue to detect an EZ within the brain of the subject. The one or more processors may store, using one or more data structures, an association between the subject and the plurality of coordinates.

In some embodiments, the one or more processors may provide, for presentation via a user interface, an output based on the plurality of coordinates identifying the corresponding plurality of locations at which to place the plurality of SEEG electrodes on the brain tissue of the subject. In some embodiments, the one or more processors may receive the dataset comprising the neuroimage in accordance with at least one of a plurality of modalities. The plurality of modalities may include a positron emission tomography (PET), magnetic resonance imaging (MRI), electroencephalography (EEG), single-photon emission computed tomography (SPECT), or magnetoencephalography (MEG). In some embodiments, the one or more processors may receive the dataset comprising a metric indicating a degree of neuropsychological function in accordance with at least one of a plurality of neuropsychological evaluations. The plurality of neuropsychological evaluations may include a verbal test, a visuospatial memory test, a picture naming test, a phonemic fluency test, a semantic fluency test, or a word reading test. In some embodiments, the one or more processors may receive the dataset including at least one of (i) a plurality of traits, (ii) a clinical history, (iii) an epilepsy history, or (iv) a plurality of semiology characteristics of the subject.

In some embodiments, the one or more processors may generate the plurality of coordinates by generating a plurality of trajectories for the corresponding plurality of locations. Each trajectory of the plurality of trajectories may define at least one of an angle or a depth of placement for the SEEG electrode of the plurality of SEEG electrodes. In some embodiments, the one or more processors may generate the plurality of coordinates by determining, for each of the plurality of coordinates, a classification of a plurality of classifications. The plurality of classifications may include at least one of a mesial/lateral temporal (MLT) region, temporal/basal/occipital (TBO) region, anterior perisylvian (AP) region, or perisylvian (P) region.

In some embodiments, SEEG data corresponding to brain activity used to detect the EZ in the brain may be measured via the plurality of SEEG electrodes placed along the brain tissue at the plurality of locations identified by the plurality of coordinates. In some embodiments, an anatomical site of the brain tissue to be placed with the plurality of SEEG electrodes may correspond to at least one of an amygdala, a head of hippocampus, a tail of hippocampus, an entorhinal cortex, a fusiform gyrus, a frontal pole, a temporal pole, an orbitofrontal cortex, a lingual gyrus, a pars triangularis, an inferior precentral gyrus, an inferior postcentral gyrus, a planum temporale, a cuneus, an anterior cingulate, or a posterior cingulate. In some embodiments, the ML model may be established using a plurality of examples. Each example of the plurality of examples may be for a respective subject at risk of or diagnosed with epilepsy. Each example of the plurality of examples may include: (i) a respective dataset including a respective neuroimage of a respective brain of the respective subject, and (ii) a respective plurality of coordinates for a respective plurality of locations along a respective brain tissue of the respective subject. Each of the respective plurality of coordinates may identify a respective location of the respective plurality of locations at which to place a respective SEEG electrode on the respective brain tissue to detect a respective EZ within the respective brain.

Other aspects of the present disclosure are related to systems and methods for training models to identify locations to place stereo-electroencephalography (SEEG) electrodes on brain tissues of subjects for detection of epileptogenic zones (EZs). One or more processors may identify an example from a plurality of examples. Each example of the plurality of examples may correspond with a first subject at risk of or diagnosed with epilepsy. Each example of the plurality of examples may include: (i) a first dataset including a neuroimage of a first brain of the first subject, and (ii) a first plurality of coordinates for a first plurality of locations along a first brain tissue of the first subject. Each of the first plurality of coordinates may identify a respective location of the first plurality of locations at which to place a corresponding SEEG electrode of a first plurality of SEEG nodes on the brain tissue to detect an EZ within the brain. The one or more processors may apply at least the first dataset of the example to a machine learning (ML) model comprising a plurality of parameters. The one or more processors may update one or more of the plurality of parameters of the ML model, based on applying at least the dataset of the example to the ML model. The one or more processors may store the plurality of parameters of the ML model to be used to generate a second plurality of coordinates for a second plurality of locations to place a second plurality of SEEG electrodes along a brain tissue of a second subject.

In some embodiments, the one or more processors may identify the example by selecting the example from a second plurality of examples different from the plurality of examples. Updating the one or more of the plurality of parameters of the ML model may include generating at least one tree in the ML model including a random forest using the first dataset and the first plurality of coordinates, in response to applying the example to the ML model. In some embodiments, the one or more processors may apply at least one the dataset of the example to the ML model by applying the first dataset of the example to the ML model to generate a second plurality of coordinates for a third plurality of locations to place a second plurality of SEEG electrodes along the brain tissue of the first subject. The one or more processors may update the one or more of the plurality of parameters by updating the one or more of the plurality of parameters based on a comparison between the first plurality of coordinates and the second plurality of coordinates.

In some embodiments, the neuroimage is in accordance with at least one of a plurality of modalities. The plurality of modalities may include a positron emission tomography (PET), magnetic resonance imaging (MRI), electroencephalography (EEG), single-photon emission computed tomography (SPECT), or magnetoencephalography (MEG). In some embodiments, the dataset may include a metric indicating a degree of neuropsychological function in accordance with at least one of a plurality of neuropsychological evaluations. The plurality of neuropsychological evaluations may include a verbal test, a visuospatial memory test, a picture naming test, a phonemic fluency test, a semantic fluency test, or a word reading test. In some embodiments, the first dataset includes at least one of (i) a plurality of traits, (ii) a clinical history, (iii) an epilepsy history, or (iv) a plurality of semiology characteristics of the first subject. In some embodiments, the first plurality of coordinates may include a plurality of trajectories for the corresponding first plurality of locations, each trajectory of the plurality of trajectories defining at least one of an angle or a depth of placement for the SEEG electrode of the first plurality of SEEG electrodes. In some embodiments, the first dataset may include, for each of the first plurality of coordinates, a respective classification of a plurality of classifications, wherein the plurality of classifications comprises at least one of a mesial/lateral temporal (MLT) region, temporal/basal/occipital (TBO) region, anterior perisylvian (AP) region, or perisylvian (P) region.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIGS. 1A-D: Classification of temporal lobe epilepsy SEEG implantation hypotheses. Talairach grids demonstrating implantation schemes in an example patient for each classification. Inset: 3D representation of an idealized left-sided implantation for each hypothesis demonstrating orthogonal lateral-to-medial trajectories and interrogation of cortical and subcortical structures. A: Mesial lateral temporal: Epileptogenic zone is hypothesized to be in the mesial and/or lateral compartments of the temporal lobe with implantation focused on the hippocampal formation, entorhinal cortex, perirhinal cortex, amygdala, temporal pole, and lateral temporal neocortex. B: Temporal basal occipital: This hypothesis anatomically involves the posterior and basal temporal regions with extension to the occipital lobe. In addition to temporal coverage, the implantation covers posterior inferior temporal gyrus, fusiform gyrus, occipital lobe, temporo-parietal-occipital (TPO) junction, retrosplenial cortex, and posterior cingulate cortex. C: Anterior perisylvian: The epileptogenic zone involves the paralimbic regions including anterior temporal lobe, rostral insula and orbitofrontal regions and the SEEG covers these regions. D: Perisylvian: These hypotheses are characterized by the involvement of the anterior and posterior perisylvian regions, the TPO junction, and parietal lobes. SEEG coverage extends through the temporal lobe, parietal lobe, TPO junction, and frontal and occipital regions as necessary.

FIGS. 2A-C: Semiology and electrode locations by classification. Red outline delineates the ten features with the most predictive power in differentiating classifications by recursive feature elimination (RFE). Classification schemes were validated by determining the overall accuracy of random forest classification with the features determined by RFE analysis. A: Semiology—RFE Analysis. The classification was developed using AEC features of various patients with temporal lobe epilepsy. RFE analysis demonstrates accordance with qualitatively. differentiating features (for instance visual phenomena in temporal/basal/occipital or facial/oral automatisms in anterior perisylvian). The Bernoulli Naïve Bayes classifier (α=0.001) class weighted-average precision 65.9% and recall 65% in the main cohort and precision 66.7% and recall 58.3% in the validation cohort. Individual patterns are reported in FIG. 11. B: Electrode location. Bubble plots of median (dot) and IQR (bubble) of target contact coordinates (MNI), displayed over the MN152 brain surface. Note the high degree of consistency between electrode targets. Electrode locations were extracted from post SEEG placement CT scans and registered to preoperative patient MRI and then to the MNI atlas. The Talairach and MNI coordinates of the medial most contact in each electrode were calculated for each patient. As electrodes are implanted orthogonally to the dorsolateral cranial surface, this fixes the position of the electrode, with minor changes in entry point to prevent vascular collisions. Brodmann areas were extracted in a semi-automated fashion as described in the methods. Coordinates are described quantitatively in Supplementary Table 2. C: Electrode location-RFE analysis successfully identified features predictive of each classification. These were expectedly largely extratemporal locations and corresponded to the anatomic localization of each hypothesis, e.g. frontal regions for anterior perisylvian, occipital regions for temporal/basal/occipital, etc. Bernoulli Naïve Bayes classifier (α=0.01) achieved class weighted-average precision 86.8% and recall 85% in the main cohort and precision 77.4% and recall 66.7% in the validation cohort. Bottom x-axis—Brodmann regions, Top x-axis—areas grouped by region. Individual patterns are reported in FIG. 12.

FIG. 3: Frequency of semiology-location pairs by classification. The frequency of classification by pairs is demonstrated and shows strong clustering. This clustering largely corresponds to the prior RFE/random forest classification. Most pairs were dominated by one classification with mixed colors indicating that 2 classifications had equal frequencies. Blank cells indicate that no classification had greater than 40% frequency.

FIG. 4: Resection patterns by classification. 49 of 60 patients underwent resection with an overall one-year seizure freedom rate of 73% (temporal: 86%, TBO: 71%, anterior perisylvian: 75%, perisylvian 68%, p=0.9). Each row represents an individual patient while filled cells indicated resected regions, colored by classification as in FIG. 1. Temporal (N=7): All patients had resections entirely confined to the temporal lobe and largely had preservation of posterior temporal regions and the hippocampal tail. Temporal basal occipital (N=7): 5 patients (57%) resections confined to the temporal lobe, including posterior regions such as fusiform gyrus (BA37) and ITG (BA20) with 3 (43%) undergoing resections of occipital with or without temporal lobe regions. Anterior perisylvian (N=16): 10 (63%) patients underwent resection of largely anterior temporal regions while 6 (36%) underwent resection of additional inferior frontal lobe structures. Perisylvian (N=19): 13 (68%) underwent resection of temporal regions, including posterior regions, while 6 (32%) underwent additional resection of frontal/parietal/occipital regions.

FIG. 5: Neuropsychological outcomes. Six-month neuropsychological outcomes in patients who underwent surgical resection or ablation of the EZ by side of intervention. Verbal memory (RAVLT delayed recall) declined in 26% of patients overall (36% left, 11% right), and improved in 13% (14% left, 11% right). Visuospatial memory (Rey-Osterrieth Complex Figure delayed recall) declined in 8% of patients (13% left, 0% right), and improved in 24% (19% left, 33% right). Picture naming (Boston Naming Test/NAB Naming Test) declined in 19% of patients (29% left, 0% right), and improved in 9.5% (0% left, 29% right). Phonemic fluency declined in no patients. Semantic fluency declined in 29% of patients (38% left, 13% right). Word reading (Wechsler Test of Adult Reading) declined in 9.5% (15% left, 0% right).

FIGS. 6A-C: Temporal pole SEEG trajectories—The inferior trajectory explores the basal/inferior aspects of the temporal pole while the super trajectory explores the superior temporal pole, paralimbic regions, and orbitofrontal cortices. A: Brodmann cytoarchitectonic map, B: Talairach grid representation, C: 3D reconstruction with superimposed Talairach grid.

FIGS. 7A-D: Amygdala, hippocampus, entorhinal cortex SEEG trajectories. A: Brodmann cytoarchitectonic map, B: Talairach grid representation, C: 3D reconstruction with superimposed Talairach grid of the amygdala and entorhinal cortex trajectories, D: 3D reconstruction of the hippocampal tail trajectory.

FIG. 8: Typical electrode trajectories (coronal view). Bubble plots on the MNI152 T1-weighted coronal MRI demonstrating typical orthogonal implantation that permits consistent recording from both dorsolateral cortical surfaces and medial structures including the mesial temporal lobe and subcortical structures. The cyan lines and red circles represent the respective trajectories and entry points. OFC: orbitofrontal cortex, IFG: inferior frontal gyrus, ant: anterior, Pars t: Pars triangularis, Ins: insula, pOFC: posterior orbitofrontal cortex, STG: superior temporal gyrus, PP: planum polare, PrCG: precentral gyrus, Hippo: hippocampus, PoCG: postcentral gyrus, PT: planum temporale, gyr: gyrus, post: posterior.

FIGS. 9A-J: Case 1—Anterior Perisylvian Hypothesis. A: Talairach grid demonstrating right sided implantation with standard electrode names. Note that the template anterior perisylvian implantation was expanded frontally due to the proposed hypothesis. B: Coronal T2 FLAIR image demonstrating nonspecific white matter hyperintensity in the frontal lobe. C: MEG dipoles in posterior insula and operculum overlaid over axial T1 MRI image. D: Ictal SPECT image (19 seconds after ictus) demonstrating subcortical and posterior insula uptake. E: Interictal PET with slightly decreased R temporal uptake. F: Interictal scalp video EEG with right frontotemporal sharp waves. G: Ictal scalp video EEG with typical right frontotemporal onset and bilateral generalization. H: SEEG interictal activity. SEEG evaluation showed interictal activity of spikes localized to the lateral superior temporal gyrus (J lateral electrode contacts) (Blue Arrow), at time synchronous with the mid ventral insula (T mesial contacts as well as U mid electrode contacts), mid to posterior superior temporal gyrus (T and U lateral electrode contacts) and anterior temporal regions (I mesial contacts) (blue dotted arrow). I: Ictal SEEG demonstrated huge spikes over the superior temporal gyrus, temporal pole and mesial mid ventral insula followed by low voltage fast activity over the anterior temporal poles regions (I, T and J) followed by posterior superior temporal gyrus (U lateral electrodes). J: Direct cortical stimulation using 50 Hz stimulation of J lateral contacts triggered the typical stereotypical seizures but not with the U lateral contacts.

FIGS. 10A-G: Case 2 Perisylvian vs. temporal basal occipital with a periventricular nodular heterotopia. A: Talairach grid demonstrated left-sided perisylvian vs. temporal basal occipital implantation with standard electrode names. B: Sagittal and coronal preoperative T1 MRI demonstrated extensive left periventricular nodular heterotopia extending from left atrium to temporal pole. C: Interictal MEG demonstrating left posterior temporal/basal temporal dipoles. D: Ictal SPECT (15 second injection) demonstrates left posterior temporal uptake. E: Interictal phase I scalp EEG demonstrates left posterior temporal polyspikes. F: Ictal scalp EEG demonstrates left posterior temporal onset seizures. G: Ictal SEEG demonstrates diffuse fast activity followed by delta slowing with low voltage fast activity in the posterior basal and temporal lobes (F′, O′, D′, V′ middle/lateral) spreading to the inferior temporal gyrus (E′ lateral) and cingulate gyrus (X′ medial) as well as STG (A′, B′ lateral).

FIG. 11: Semiological features by subject. Each row represents an individual subject (N=60 total), organized by proposed classification. Filled cells represent observed semiology in each subject's seizures, as determined by the treating epileptologist and/or the multidisciplinary epilepsy patient management conference.

FIG. 12: Electrode coverage by subject. Each row represents an individual subject (N=60 total), organized by proposed classification. Filled cells represent electrode coverage in that Brodmann area (bottom x-axis) and larger region (top x-axis). Note consistent coverage of temporal regions but variable coverage of extratemporal regions by hypothesis.

FIG. 13: Prediction of electrode coverage utilizing semiological features only. While the implantation hypotheses and corresponding patterns of implantation are developed using a combination of seizure semiology, anatomy, and ancillary testing (SPECT, MEG, neuropsychological evaluation, etc.), the degree to which semiology alone could predict the pattern of implantation was examined. Using a random forests classifier (Number of estimators: 200, no maximum depth, minimum samples per split: 10, minimum samples per leaf: 1), the seizure semiologies in FIGS. 2A-C were mapped to the electrode implantation (by Brodmann Area) in the original cohort (N=60) and validated this classifier in the test dataset of 12 subjects (pictured). Overall, the classifier showed significant predictive accuracy in the training dataset (precision 84.1%, recall 82.4%, F1 micro 83.2%) and moderate accuracy in the validation cohort (precision 64.7%, recall 71.1%, F1 micro 67.7%). In the validation dataset, precision was similar in all classifications (TBO: 68.4%, AP: 64.7%, P: 64.1%), but recall was best in the anterior perisylvian (TBO: 59.1%, AP: 82.4%, P: 65.1%).

FIG. 14 is a block diagram of a system for identifying locations to place stereo-electroencephalography (SEEG) electrodes on brain tissues of subjects for detection of epileptogenic zones (EZs), in accordance with an illustrative embodiment.

FIG. 15 is a block diagram of a process of training a machine learning (ML) model to identify locations to place SEEG electrodes on brain tissues of subjects for detection of EZs, in accordance with an illustrative embodiment.

FIG. 16 is a block diagram of a process for applying a model to identify locations to place SEEG electrodes on brain tissues of subjects for detection of EZs, in accordance with an illustrative embodiment.

FIG. 17 is a block diagram of a process to produce outputs in the system for identifying locations to place SEEG electrodes, in accordance with an illustrative embodiment.

FIG. 18 is a flow diagram of a method of training a machine learning (ML) model, in accordance with an illustrative embodiment.

FIG. 19 is a flow diagram of a method of identifying locations to place SEEGs, in accordance with an illustrative embodiment.

FIG. 20 depicts a block diagram of a server system and a client computer system, in accordance with an illustrative embodiment.

### DETAILED DESCRIPTION

Following below are more detailed descriptions of various concepts related to, and embodiments of, systems and methods for identifying locations to place stereo-electroencephalography (SEEG) electrodes on brain tissues of subjects for detection of epileptogenic zones (EZs). It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes semiology-driven framework for explorations of temporal lobe epilepsy.

Section B describes systems and methods for identifying locations to place stereo-electroencephalography (SEEG) electrodes on brain tissues of subjects for detection of epileptogenic zones (EZs).

Section C describes a network environment and computing environment which may be useful for practicing various computing related embodiments described herein.

### A. Semiology-Driven Framework for Explorations of Temporal Lobe Epilepsy

### Introduction

Objective: Temporal lobectomy is effective for drug-resistant temporal lobe epilepsy (TLE), but broad resections risk neuropsychological morbidity. Stereoelectroencephalography (SEEG) supports hypothesis-driven exploration of seizure networks, where semiology, together with imaging, scalp EEG, and neuropsychology, anchors implantation strategies without serving as a sole classifier. Common SEEG implantation patterns in TLE were defined from a stereotaxic functional-anatomical perspective and their reproducibility was tested.

Methods: 72 patients (60 main, 12 validation) with drug-resistant temporal lobe epilepsy (TLE) who underwent SEEG were analyzed. Implantation strategies converged into four reproducible patterns-mesial-lateral temporal, temporal-basal-occipital, anterior perisylvian, and perisylvian-guided primarily by non-invasive data with semiology exerting the greatest influence. Seizure semiology, neuroimaging, and electrophysiological recordings were systematically collected. To evaluate the reproducibility of implantation patterns, machine learning classifiers (Naïve Bayes, Random Forests) were trained to predict implantation classification based on semiological features and electrode coverage. Surgical and neuropsychological outcomes were prospectively assessed at 1 year.

Results: Classification based on semiology alone achieved moderate accuracy in predicting outcomes (precision 66%, recall 65%). Electrode coverage provided substantially higher predictive power (precision 87%, recall 85%). Crucially, semiological features showed strong concordance with the actual electrode implantation patterns (precision 84%, recall 82%), demonstrating that semiology functions as a reproducible heuristic for guiding implantation strategy. Following SEEG, 94% of patients proceeded to definitive therapy (82% tailored resection, 12% neuromodulation), with a one-year seizure freedom rate of 73%, independent of lesion status or implantation category. Postoperatively, 26% of resection patients demonstrated verbal memory decline, more frequently after left-sided (38%) than right-sided (7.7%) interventions.

Significance: Reproducible SEEG implantation patterns in temporal lobe epilepsy are characterized, viewed from a stereotactic functional-anatomical perspective and informed by semiology. Computational analyses were used to examine the consistency of these patterns, offering insights into how implantation strategies relate to network localization and potential surgical approaches.

### Points

Temporal lobectomy remains the standard surgical treatment for drug-resistant temporal lobe epilepsy (TLE), but reliance on broad anatomical templates raises ongoing concerns regarding seizure freedom and neuropsychological morbidity. Reports describing SEEG implantation strategies in TLE are scarce and heterogeneous. In the absence of standardized anatomical frameworks, implantation often varies widely across centers, making anatomo-clinical interpretation and reproducibility challenging.

Reproducible stereo-anatomical patterns of SEEG implantation-mesial/lateral temporal, temporal-basal-occipital, anterior perisylvian, and perisylvian-grounded in semiology-driven hypothesis generation and consistent with the core principles of SEEG methodology are defined herein. Semiology was used as the primary determinant of implantation trajectories, complemented by anatomical and functional considerations, and machine learning was applied to test the consistency and predictability of these patterns across patients. By systematically grouping and categorizing common implantation schemas, this framework strengthens anatomo-clinical interpretation and provides a rational, reproducible approach to SEEG exploration in TLE, with direct implications for surgical planning and outcome optimization.

### Context

Despite the long-standing success of temporal lobectomy for drug-resistant temporal lobe epilepsy (TLE), surgical strategies remain constrained by broad anatomical dichotomies-mesial versus lateral, or temporal versus temporal-plus. These frameworks oversimplify the functional-anatomical complexity of temporal and perisylvian networks and provide limited guidance for how stereo-electroencephalography (SEEG) implantations should be structured to interrogate seizure dynamics in individual patients. In this context, while SEEG has become central to presurgical evaluation in difficult-to-localize epilepsy, few systematic frameworks currently describe how non-invasive data-particularly ictal semiology- are incorporated into implantation strategies.

Although SEEG was introduced as a methodology grounded in anatomo-electroclinical correlation, with electrode trajectories designed to test explicit clinical hypotheses, there are no systematic descriptions of how implantation strategies should be organized in TLE. The importance of exploring perisylvian and paralimbic structures in addition to mesial temporal regions was emphasized. Other approaches have defined concepts such as “temporal-plus” or basal temporal epilepsies. Yet, these approaches have primarily emphasized seizure classification and clinical outcomes rather than codifying implantation geometry. What remains lacking is a systematic translation from non-invasive data—such as ictal semiology—into specific anatomical targets and stereotactic coordinates to be sampled with SEEG electrodes. Reports of electrode trajectories are generally schematic, center-specific, and heterogeneous, and they often lack the granularity required to reproduce implantation strategies across institutions.

In this context, there is little framework to link non-invasive data features, in particular, ictal semiology, with reproducible implantation patterns. Other approaches remain lesion- or imaging-driven, with semiology treated as a secondary or supportive observation rather than as a primary determinant of electrode placement. This is at odds with the core principles of SEEG methodology, which emphasize hypothesis-driven implantation based on clinical semiology, orthogonal trajectories to optimize safety and coverage, and systematic sampling of cortical-subcortical networks to interrogate seizure onset and propagation. Without consistent adherence to these principles, implantation strategies vary substantially between operators and centers, making the interpretation of anatomo-clinical patterns more challenging and limiting the ability to draw generalizable conclusions from SEEG studies.

The aim is to address this gap by characterizing reproducible SEEG implantation patterns in temporal lobe epilepsy, framed from a stereotactic functional-anatomical perspective and informed by semiology alongside other clinical data. SEEG is fundamentally grounded in anatomo-electroclinical hypothesis testing, where semiology provides an initial anchor that is refined by imaging, neuropsychology, and electrophysiology. Computational methods were used to examine the consistency of these implantation strategies, offering insights into how semiology relates to functional-anatomical schemas and how such frameworks may optimize surgical approaches to maximize seizure freedom and minimize neuropsychological morbidity.

### Methods

### Patient Selection

A cohort of consecutive patients who underwent stereoelectroencephalography (SEEG) for suspected drug-resistant temporal lobe epilepsy (TLE) were collected. A total of 75 patients underwent SEEG for drug-resistant epilepsy during the study period. Inclusion criteria were: (1) age ≥18 years, and (2) a pre-implantation clinical hypothesis implicating the temporal lobe. Patients were excluded if they lacked sufficient clinical follow-up (<1 year). To reduce confounds related to variability in implantation geometry, only orthogonally oriented electrode trajectories were included in the analysis. 60 patients were included in the main cohort. The principal variable of interest-implantation hypothesis-was prospectively formulated during multidisciplinary epilepsy surgery planning conferences but was retrospectively extracted and classified for analysis, incorporating past literature and approaches but enhanced by the authors' experience.

Electrode implantations were planned through a consensus that incorporated all available non-invasive data; however, a particular emphasis was placed on early and prominent ictal semiological features and their anatomical correlates, as semiology is a primary driver of anatomo-electroclinical hypotheses. Structural and functional imaging and Phase I electrophysiology were also considered as complementary factors in refining electrode placement. Classification was derived from the most frequently observed implantation patterns during the mature phase of SEEG clinical practice and was subsequently confirmed through consensus among the authors.

To examine the consistency of these implantation strategies and to assess model generalizability with out-of-sample performance, a prospective validation cohort was subsequently assembled using identical inclusion and exclusion criteria. The validation cohort included 12 consecutive patients who underwent SEEG implantation between 2023 and 2024. This cohort was held out entirely from training and feature selection and was used exclusively for external validation of the machine learning classifiers.

### Data Collection

Demographic information, epilepsy history, and neuroimaging data were extracted from the electronic medical record. Seizure semiology was compiled from multiple sources: the initial clinical history provided by the treating epileptologist, ictal behavioral observations during video-EEG monitoring, formal interpretation by the epilepsy monitoring unit (EMU) team, multidisciplinary epilepsy conference notes, and independent re-review of ictal recordings by two authors (TA and JGM).

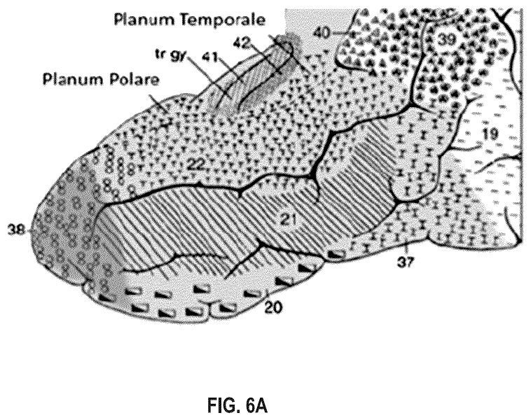

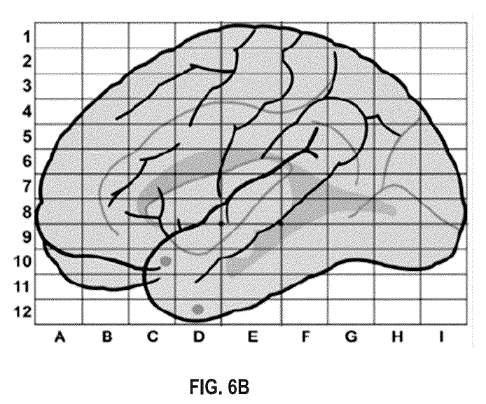

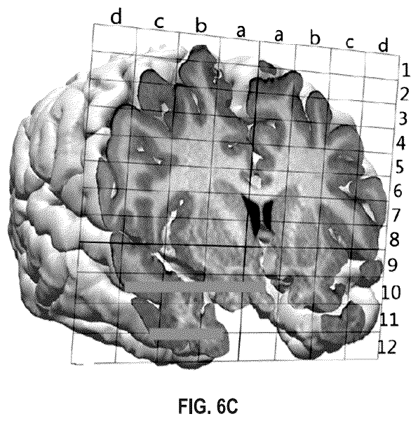

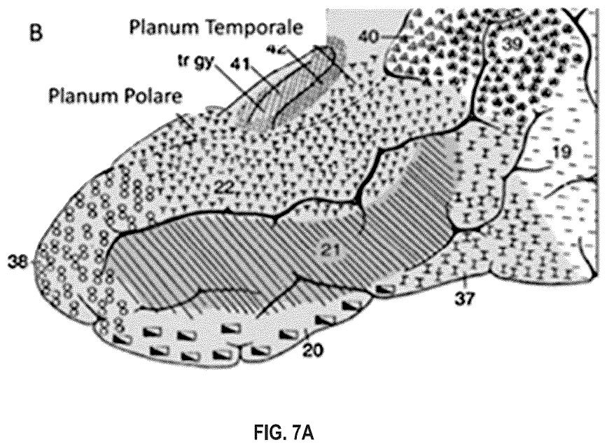

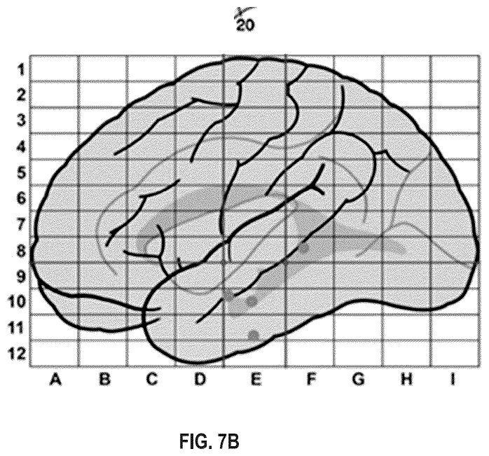

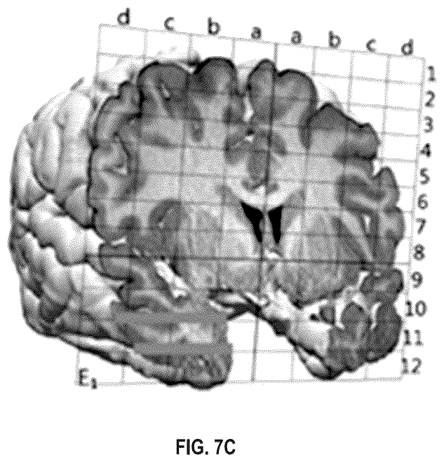

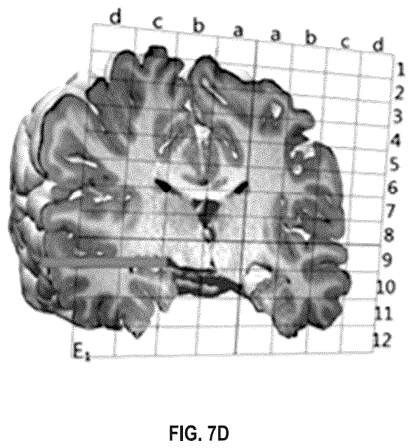

Surgical outcomes, neuropsychological performance, and perioperative complications were also retrieved from the clinical record and confirmed during in-person follow-up visits. Seizure freedom was defined as Engel Class I. Additional methodological details regarding Phase I presurgical evaluation, neuropsychological testing, electrode implantation strategy, electrode localization, resection segmentation, and surgical technique are provided in the Supplementary Methods and illustrated in FIGS. 6A-8.

### Feature Selection and Machine Learning Classification

The consistency of this approach was quantitatively evaluated using two strategies applied in two cohorts of patients. In both cases, the machine learning model was trained on the main dataset (N=60) and tested in the validation cohort (N=12). First, the degree to which seizure semiology, electrode location, and resection region could predict the hypothesis classification were determined at the subject level. To identify the most relevant features, a random forest (Gini criterion, 100 trees, 1 sample per leaf, 2 samples per split)-based recursive feature elimination (RFE) was used for feature selection to identify the 10 features with the greatest impact on classification prediction accuracy. These features were used to predict hypothesis classification. As these features were one-hot (binary) encoded, a classifier model that is optimized for this data type, the Bernoulli Naïve Bayes (BNB) classifier, was elected for use due to its simplicity. Hyperparameters (additive smoothing parameter-a) were optimized using a grid search approach.

Second, in a prospective cohort of new patients, it was aimed to determine whether SEEG implantation strategies could be predicted from semiological features alone, independent of predefined classification schemes. This design was specifically intended to mitigate the risk of circular reasoning, as the predictive input (ictal semiology) reflects clinical manifestations prior to implantation, whereas the outcome (implantation patterns) represents the subsequent surgical planning process. By maintaining a strict separation between hypothesis-generating data and the implantation strategies being tested, the likelihood that the model would simply reproduce the assumptions underlying electrode placement was reduced, the influence of site- or surgeon-specific biases was minimized, and it was ensured that semiology's predictive value was assessed transparently. In doing so, the analysis directly tests whether early clinical features contain sufficient information to explain implantation patterns, thereby supporting reproducibility and providing a framework that extends beyond individual clinical practice. As this is inherently a multiclass/multioutput classifier relating multiple semiologies to multiple cortical locations, a random forests (RF) classifier was used. Hyperparameters were optimized using a grid search approach. Final model performance was evaluated on an independent held-out validation cohort, which was never used in training or hyperparameter tuning. Machine learning was performed using scikit-learn in Python 3.10. Heatmaps and other figures were generated using seaborn.

### Statistical Analyses

Other statistical analyses were performed in R 4.2.0 (R Foundation for Statistical Computing, Vienna, Austria) with RStudio (Posit Software, Boston, MA). Tables were generated using the gtsummary package. Continuous variables are reported as median [IQR] and categorical as N (%). As appropriate, Fisher's exact test, the Kruskal-Wallis test, and the Chi-squared test were utilized for univariate statistical comparisons. P<0.05 was the prespecified threshold for significance. To ensure reproducibility and transparency, statistical and machine learning code used for data preprocessing, modeling, and figure generation is available from the corresponding author upon request.

### Results

### Cohort Characteristics

The main cohort consisted of 60 patients with drug-resistant TLE who were referred for invasive Phase II monitoring. Demographic and surgical details are reported in Table 1. The median age was 34 [28, 44] years at SEEG and 55% were male. Neither varied significantly by hypothesis (p=0.3 for both). The median number of electrodes was 14 [13, 15] and was significantly different (p=0.044) between classifications. Only 1 patient of 60 (1.7%) had additional electrodes implanted due to insufficient coverage of the EZ. The overall clinically significant complication rate from SEEG was 3.3% (2/60) and did not vary by hypothesis classification (p=0.8). Details for the validation cohort are in Supplementary Table 1.

### Classification of TLE Hypotheses and SEEG Explorations

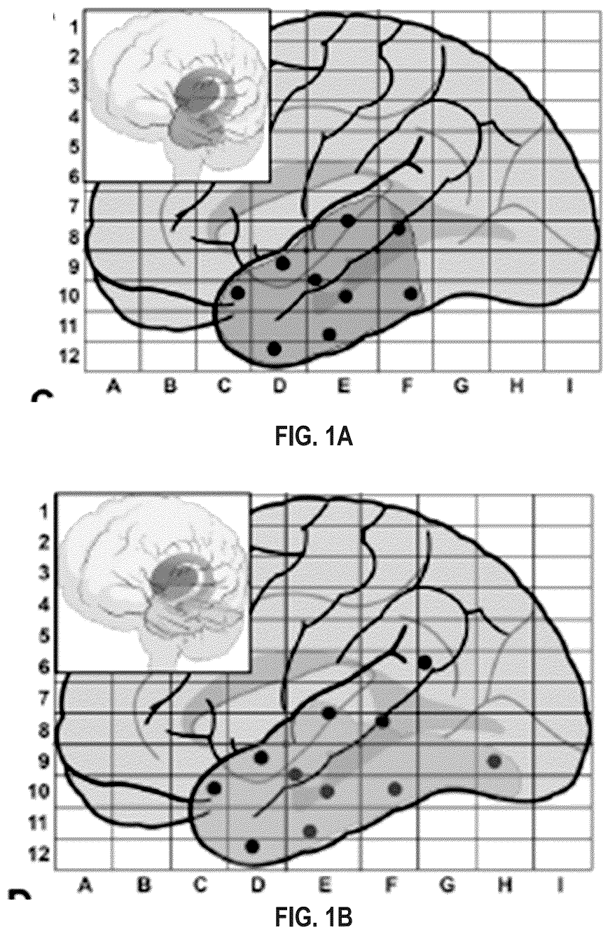

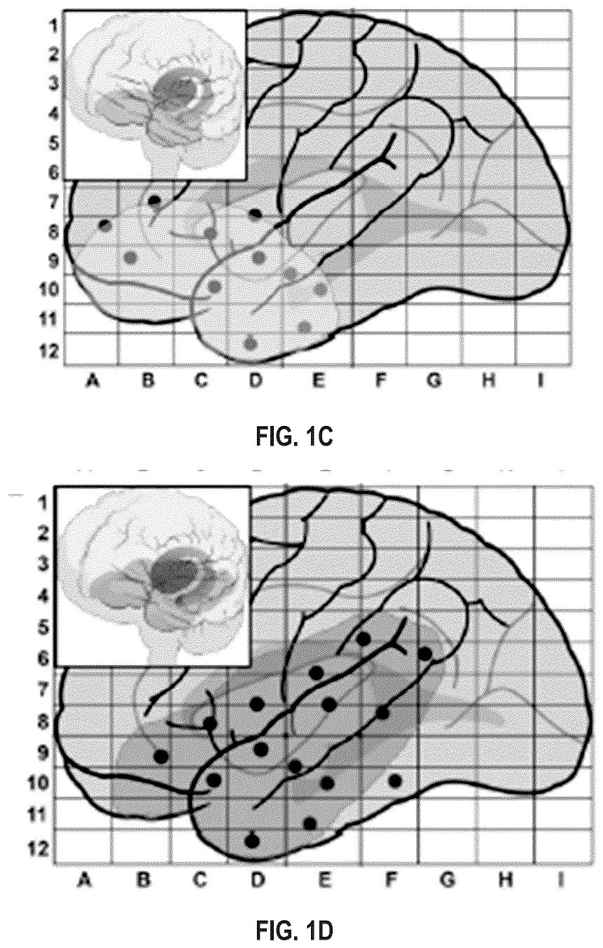

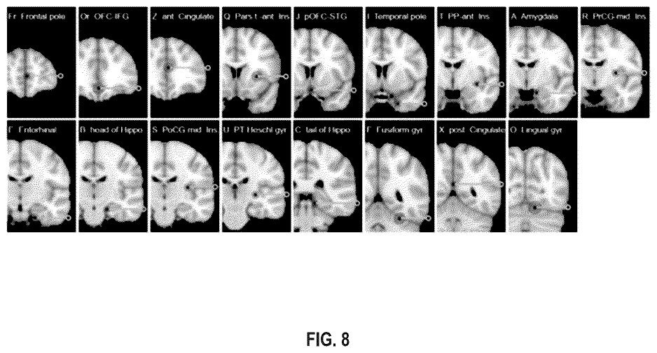

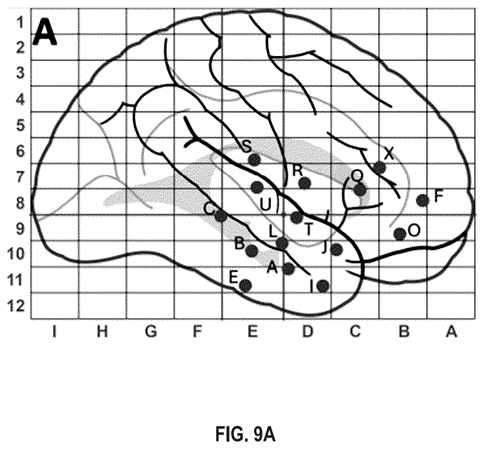

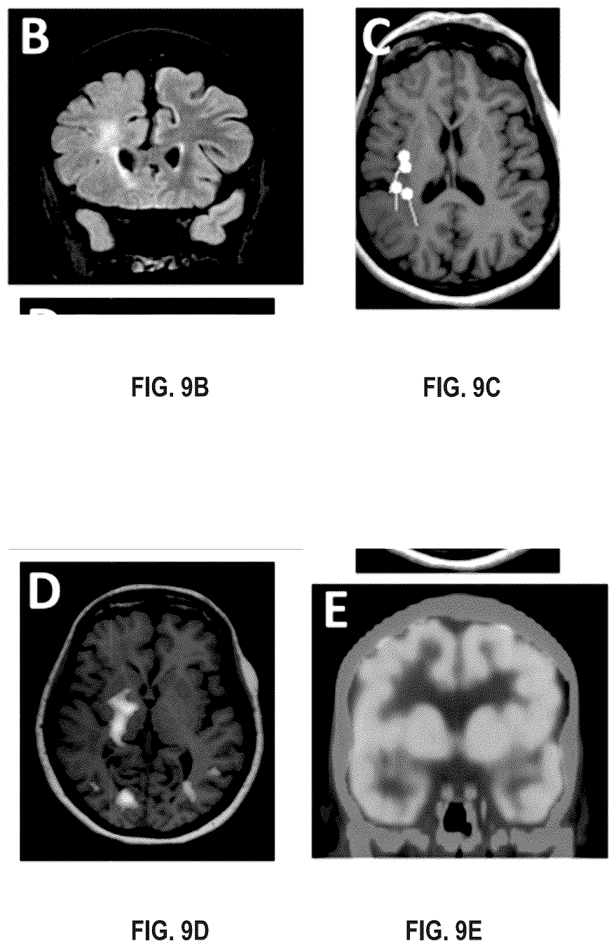

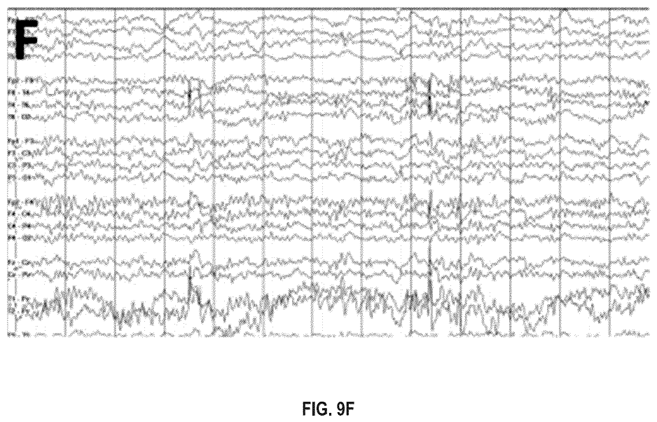

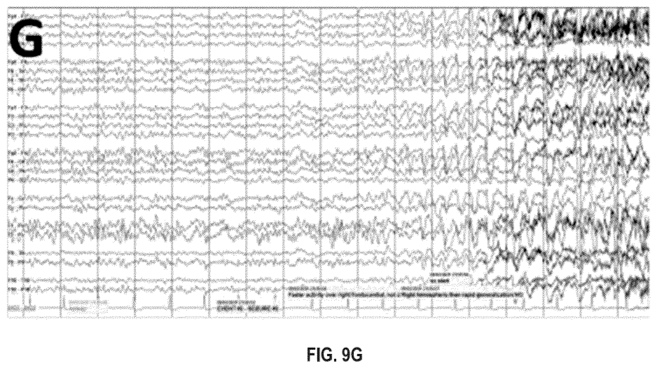

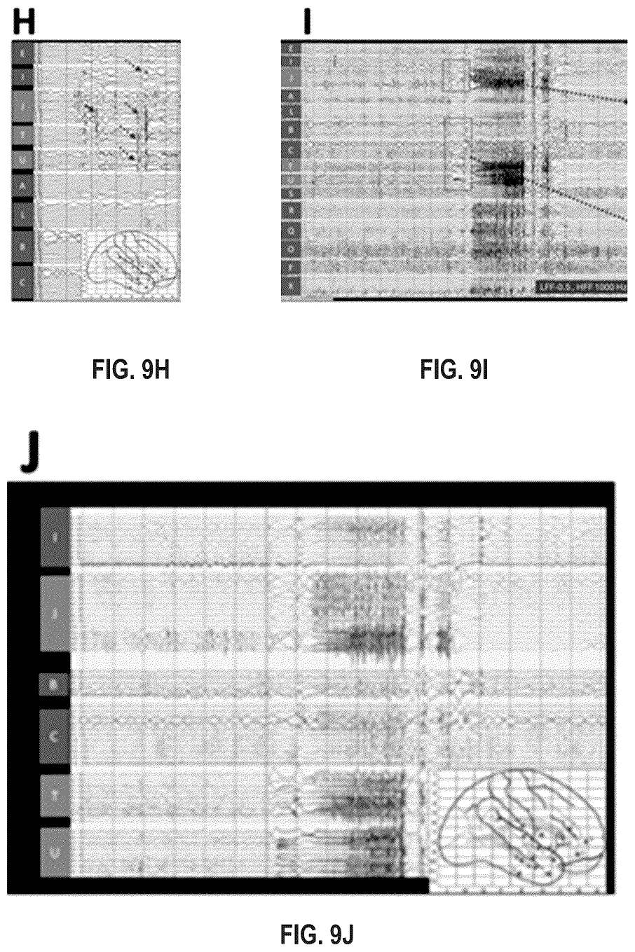

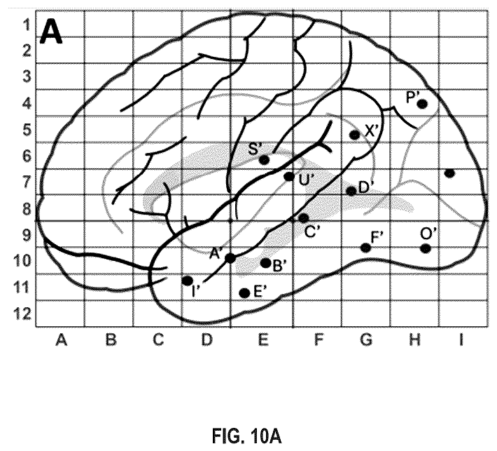

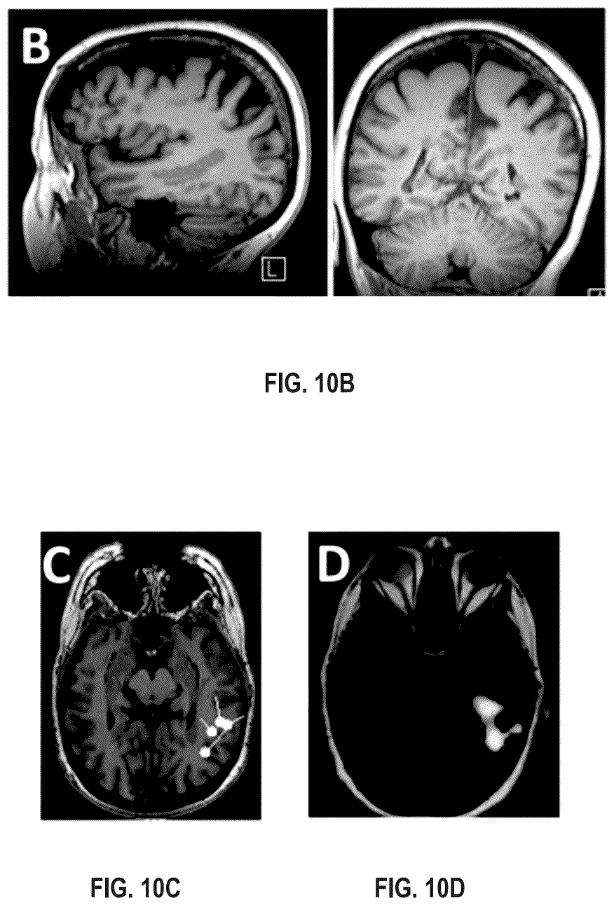

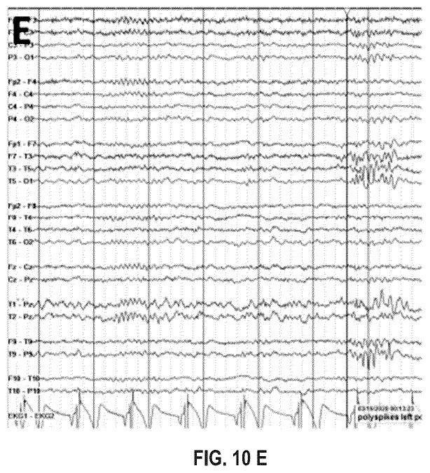

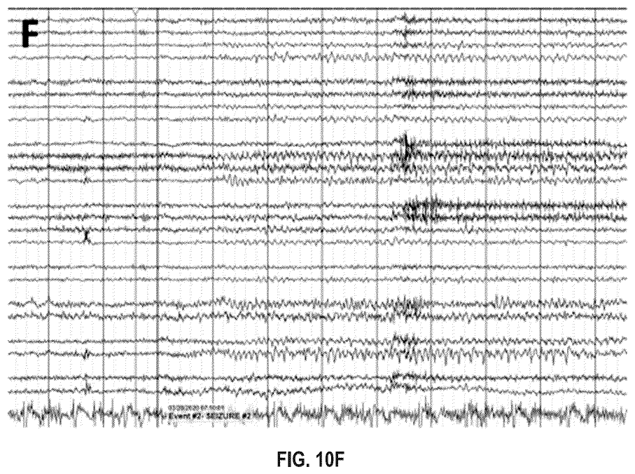

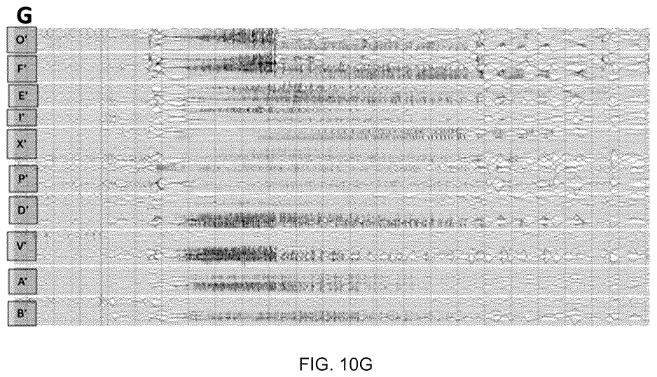

Presented herein is a preliminary and schematic classification system that organizes the most common hypothesis-driven implantation strategies into four distinct patterns: 1) Mesial/Lateral Temporal (MLT), 2) Temporal/Basal/Occipital (TBO), 3) Anterior Perisylvian (AP), and 4) Perisylvian (P). Both the cytoarchitectonic parcellation of the temporal lobe (and adjacent structures) and each patient's anatomical, semiological, and non-invasive electrophysiological data were considered. Each clinical hypothesis corresponds to a characteristic SEEG implantation pattern. As the temporal lobe is the core of each exploration, there is substantial overlap among key temporal electrodes but significant variability in the posterior extent of coverage and coverage of extratemporal structures. When multiple hypotheses are present, the resulting implantation strategy typically reflects a combination of these patterns. Detailed descriptions of each implantation pattern are provided below. Anatomic illustration and case examples are shown in FIG. 1. Additional case examples with semiological and surgical details can be found in Supplementary Results (FIGS. 9A-10G).
I). Mesial/lateral temporal (MLT): The MLT hypothesis (FIG. 1A) is proposed when the pre-implantation investigation suggested that the EZ zone is confined in the mesial/lateral compartments of the temporal lobe without significant involvement or interaction with posterior temporal or extratemporal regions. Semiologically, it is characterized by minimal ictal manifestations, potentially by autobiographic (déjà vu or dreamy state), gustatory or autonomic auras. Here, the SEEG implantation is focused on the hippocampal formation, entorhinal cortex (Brodmann Area 34, 28, 27), perirhinal cortex (BA 35, 36), amygdaloid complex, the temporal pole (BA 38) and the lateral temporal neocortex as the anterior aspects of the superior, middle, and inferior temporal gyri (BA 22, 21, 20, respectively), usually no further posteriorly than Heschl's gyrus (BA 41, 42). Both mesial and lateral temporal structures are explored concomitantly with orthogonal electrodes. Typically, a median of 11 [9, 13] electrodes were utilized to interrogate this hypothesis. Implantation patterns exploring other hypothesis types build upon the MLT pattern and include greater extratemporal coverage.
II). Temporal/basal/occipital (TBO): TBO hypotheses (FIG. 1B) can be broadly characterized by involvement of posterior and basal temporal lobe regions with extension to the occipital lobe. Non-invasive data suggest temporal lobe involvement, but with additional semiological, electrophysiological and/or imaging data suggesting the possible involvement of the basal and dorsal-lateral temporal/occipital areas. Commonly, these additional ictal signs and symptoms were associated with the presence of visual auras, conjugate eye deviation, and, if on the dominant side, language disruption. This approach extends the MLT implantation to the basal temporal lobe and fusiform gyrus (BA 37), occipital lobe (BA 17-19), temporal-parietal-occipital (TPO) junction (BA 39, 40), retrosplenial cortex (BA 29, 30), and posterior cingulate cortex (BA 23, 31). The temporal/basal/occipital explorations were achieved with 15 [14, 15] electrodes implanted orthogonally.
III). Anterior perisylvian (AP): The AP hypothesis (FIG. 1C) interrogates the involvement of the paralimbic regions in the organization of temporal lobe seizures. Typically, the exploration includes key paralimbic structures including the anterior temporal lobe (temporal pole, mesial temporal structures including the amygdala and the anterior hippocampus), rostral ventral aspects of the insula (BA 13), orbitofrontal regions (BA 11, 12), frontal pole (BA 10), inferior frontal gyrus (BA 44, 45, 47), and subgenual cingulate cortex (BA 25). Superior temporal gyrus and inferior frontal gyrus electrodes cover the temporal/frontal lobes laterally and the insula medially. If more prominent naturalistic and integrative motor manifestations are prominent, additional frontal lobe structures including premotor (BA 6, 8), anterior cingulate (BA 32, 33) are added. These cortical areas are structurally interconnected via the uncinate fasciculus, surrounding the sphenoidal surface of the Sylvian fissure, surrounding the limen insulae. Semiology can include fearful behavior, oral automatisms, autonomic symptoms, and other limbic manifestations. The AP explorations were achieved with median 14 [13, 15] orthogonal electrodes.
IV). Perisylvian (P): Perisylvian hypotheses (FIG. 1D) were characterized by the contribution of anterior and posterior perisylvian regions, possibly involving the dorsal-lateral temporo-parietal-occipital (TPO) cortex, posterior insula, and vicinity. Due to the multimodal associative characteristics of the cortical organization in this region, semiological features are complex and many times difficult to interpret. Manifestations can include dizziness, conjugate eye deviation, extremities sensory symptoms, sensory and motor face and throat manifestations, auditory phenomena and speech disturbance. SEEG sampling includes the TPO junction and associated temporal and parietal operculum (BA 39, 40), superior parietal lobule (BA 5, 7), perisylvian primary sensory cortex (BA 3, 1, 2), retrosplenial cortex (BA 29, 30), posterior cingulate cortex (BA 23, 31), perisylvian primary motor-sensory cortex (BA 4), and inferior frontal gyrus (BA 44, 45). As in AP explorations, STG and frontal electrodes can cover the insula (BA 13) medially. A median of 14 [14, 16] electrodes were used for this exploration.

### Quantitative Validation of Semiological Features

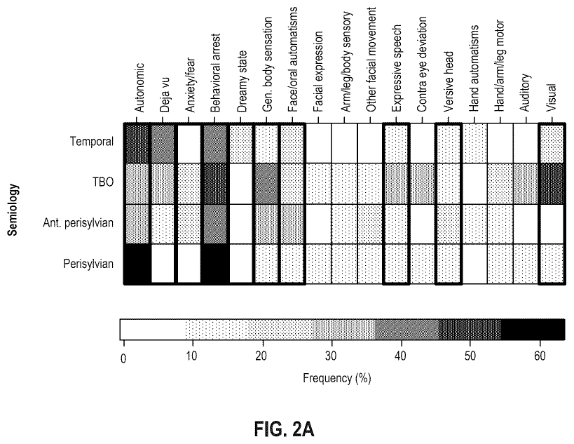

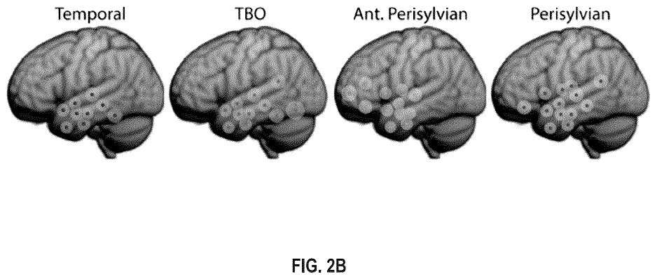

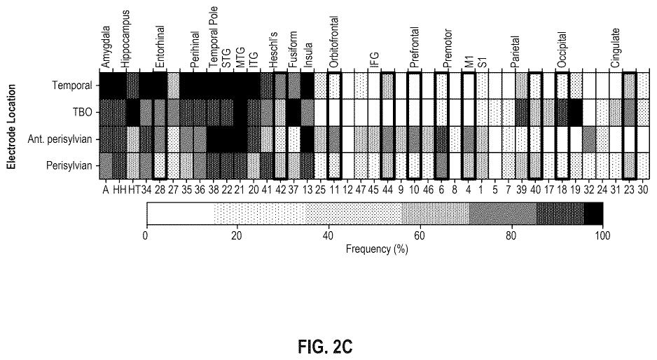

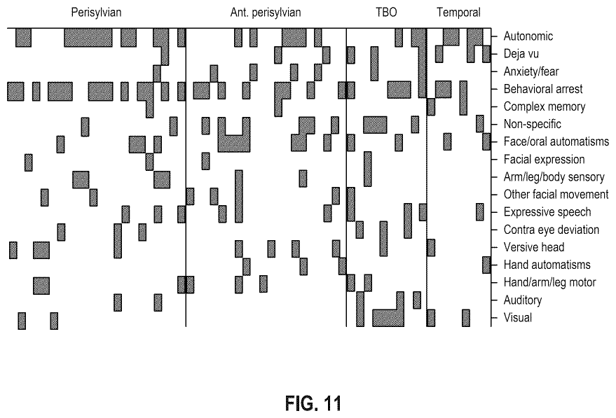

The frequencies of semiological features by hypothesis classification are reported in FIG. 2A and semiology by individual patients in FIG. 11. Using RFE, the 10 most predictive features were selected (FIG. 2A, Supplementary Table 3). This quantitative approach identified several features consistent with the conceptualization. Déjà vu (33%) and dreamy state (50%) were most common in MLT. TBO was associated with visual symptoms (56%) and expressive speech disturbances (30%). AP had high proportion of facial/oral automatisms (47%), versive head movements (40%), non-localized/generalized sensation (46%) and anxiety/fear (56%). Finally, P was associated with behavioral arrest (47%), autonomic changes (50%), versive head movements (40%), and arm/leg/body lateralized sensory changes (23%). Using the BNB classifier (α=0.001) on the top 10 selected semiological features, class-weighted average precision of 65.9% and recall of 65% were achieved in the main cohort, and 66.7% precision and 58.3% recall were achieved in the independent validation cohort. While these results reflect moderate predictive performance, they indicate that semiology alone carries meaningful discriminative information (see Supplementary Table 4 for full performance metrics).

### Electrode Locations in Stereotaxic Space

The Talairach and MNI coordinates of the most distal contact of each electrode (Supplementary Table 2) were collected to provide indirect targeting coordinates to replicate this approach. There was a high degree of consistency between implantations that corresponded with the AEC hypotheses (FIG. 2B). Typical trajectories are shown in FIG. 8.

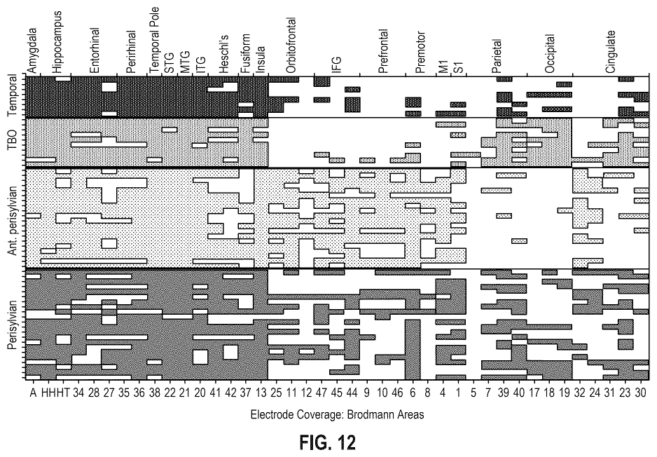

Electrode coverage is shown in aggregate in FIG. 2C and in individual subjects in FIG. 12. Using RFE, the 10 electrode locations with the most predictive ability were identified to distinguish classes (FIG. 2C, Supplementary Table 3). As the electrodes within the temporal lobe were similar between classifications, most selected electrodes were extratemporal. Specifically, occipital (90%) and posterior cingulate (80%) were most common in TBO. Prefrontal (80%), inferior frontal gyrus (85%), premotor (90%), primary motor (75%), and orbitofrontal (85%) were most common in AP. TPO/parietal (68%), posterior cingulate (68%), premotor (82%), and primary motor (64%) electrodes were most common in P. Using the BNB classifier (α=0.01) applied to electrode implantation patterns, a class-weighted average precision of 86.8% and recall of 85% was achieved in the main cohort (validation: 77.4% precision and 66.7% recall). These findings suggest that electrode coverage patterns encode substantial information relevant to hypothesis classification, though further refinement may improve consistency across cohorts.

Semiology Vs. Electrode Location

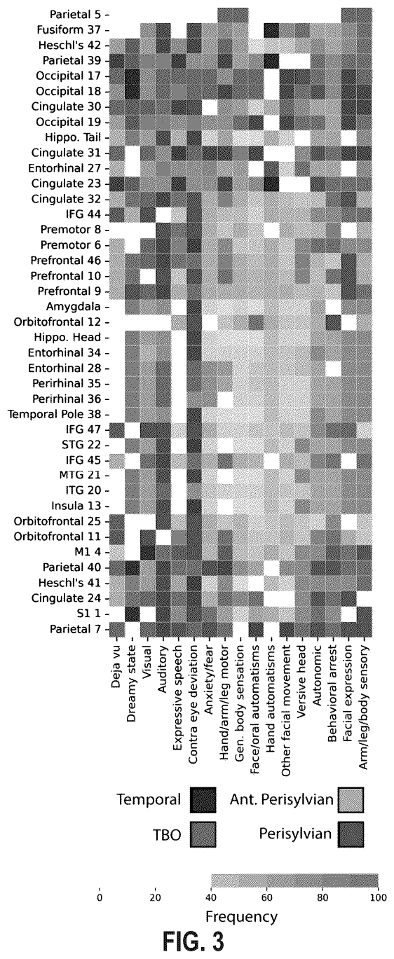

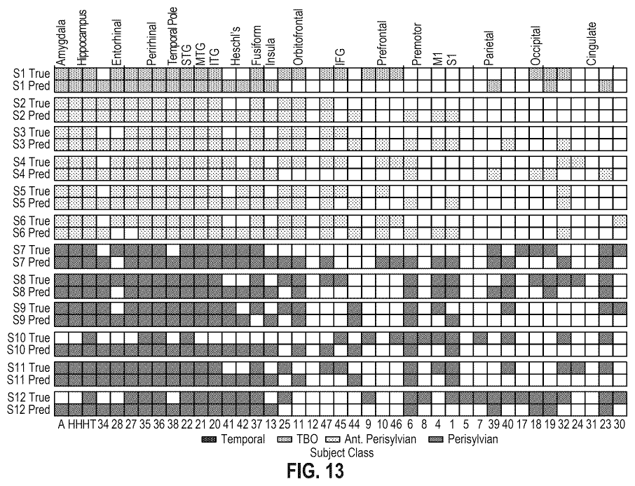

In clinical practice, AEC hypotheses and subsequent electrode coverage are determined using a combination of Phase I video EEG, neuroimaging, and neuropsychological evaluation, but with a key role of seizure semiology. To elucidate structure-semiology relationships in TLE, it was sought to predict the distribution of electrode coverage using solely semiology. Each hypothesis classification demonstrated a distinct combination of cardinal semiological features and electrode locations (FIG. 3), substantiating the hypothesis classification. Using a random forests classifier (Number of estimators: 200, no maximum depth, minimum samples per split: 10, minimum samples per leaf: 1), the seizure semiologies in FIG. 2A were mapped to the SEEG implantation (by Brodmann Area) in FIG. 2C in the original cohort (N=60) and validated this classifier by predicting the implantation pattern in the validation cohort (FIG. 13). The classifier accurately predicted patterns of temporal lobe electrode coverage and effectively captured the distribution of necessary extratemporal implantation. In the main cohort, it demonstrated strong predictive performance, with a precision of 84.1%, recall of 82.4%, and a micro-averaged F1 score of 83.2%, but more moderate in the validation cohort (precision 64.7%, recall 71.1%, F1 micro 67.7%) (see Supplementary Table 5 for additional performance metrics).

### Post SEEG Intervention

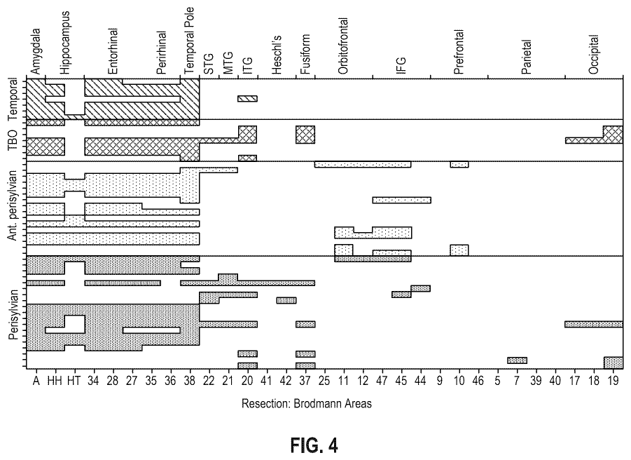

Following SEEG, 82% underwent surgical resection, 12% neuromodulation, and 6% no additional intervention. 69% of resection patients underwent tailored SEEG-guided temporal resections, while 31% underwent extratemporal with or without temporal resections. Individual resection patterns are illustrated in FIG. 4. The median resection volume was 30 [22-40] cc and did not vary significantly between classifications (p=0.4), nor did the degree of extratemporal vs. temporal resection (Table 1; p=0.3). No MLT implantations resulted in extratemporal resections. Few patients underwent a traditional “standard temporal lobectomy,” e.g. en bloc resection of the anterior temporal neocortex (typically 4.5 cm on the dominant side and 5.5 cm on the non-dominant side) along with the amygdala and hippocampus. For instance, of 49 patients, 29 (59%) had the hippocampal head resected and 12 (24%) had the hippocampal body/tail resected. Only 3/72 patients had resections that could be characterized as standard temporal lobectomies. Using the RFE feature selection approach to areas of resection (Supplementary Table 6), the overall classification accuracy using a BNB classifier (a=0.8) over all electrode coverage predicted hypothesis classification with class weighted-average precision 43.4% and recall 46.9% in the main cohort and precision 68.9% and recall 58.3% in the validation cohort

### (Supplementary Table 7)

### Seizure Outcomes

In resection patients in the main cohort (N=49), the one-year seizure freedom rate was 73% and did not vary by classification (MLT: 86%, TBO: 71%, AP: 75%, P: 68%; p=0.9), sex, age, MRI abnormality, number of electrodes, or laterality of implantation (Supplementary Table 8). One-year seizure freedom was significantly associated with post SEEG resection (100% of seizure free patients underwent resection), p<0.001). In the validation cohort, the one-year seizure freedom rate was 83% and did not vary by classification (TBO: 100%, AP: 80%, P: 83%; p=0.9), for an overall seizure freedom rate in both cohorts of 75% across 61 resection patients.

### Relationship of MRI Lesions to Implantation Patterns

MRI abnormalities suggestive of hippocampal sclerosis and focal cortical dysplasia, among others, were found in 67% of patients and did not vary by classification (p=0.3, Table 1). The presence of an MRI visible lesion does play a role in surgical planning but is secondary to the AEC hypotheses. MRI lesions did not affect seizure freedom (not seizure free—60% vs. seizure free—69%, p=0.5, Supplementary Table 8).

### Neuropsychological Outcomes

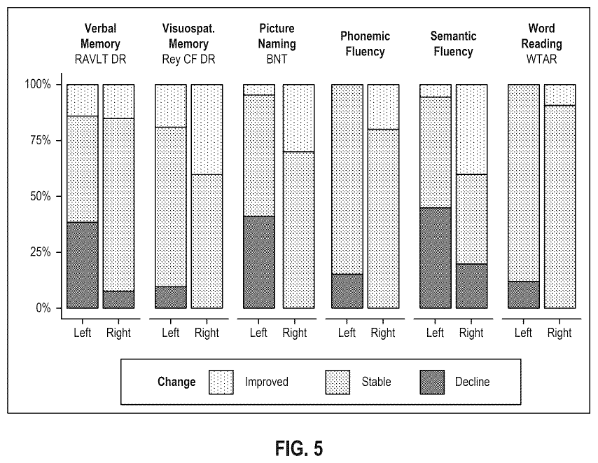

Neuropsychological testing was obtained pre-operatively in all patients as part of Phase I evaluation and for all resection/ablation patients at 6-months post-resection, with variable follow-up (25/49 in main cohort, 12/12 in validation, N=37 total). Decline/improvement is defined as change >1 S.D. from preoperative performance. Verbal memory declined in 26% of patients (38% left, 7.7% right), and improved in 15% (14% left, 15% right, L vs. R. p=0.14). Picture naming declined in 28% (41% left, 0% right), and improved in 13% overall (4.5% left, 30% right, p=0.019). Finally, visuospatial memory declined in 6.5% of patients (9.5% left, 0% right), and improved in 26% (19% left, 40% right, p=0.3) (FIG. 5). Other measures, including reading and semantic/phonemic fluency are reported in Supplementary Table 9.

### Analysis of Misclassified Subjects

Misclassified subjects were primarily due to overlapping semiological features across different hypothesis classes, ambiguous or mixed ictal manifestations, and discordances between semiology and imaging findings. Patients presenting with both mesial temporal features such as déjà vu and posterior symptoms such as visual aura were sometimes misclassified between MLT and TBO categories. Similarly, semiologies such as fear and visual phenomena, which are characteristic of AP and TBO hypotheses respectively, led to misclassification when present in combination within the same patient. In other cases, perisylvian patients with prominent sensory symptoms and behavioral arrest were predicted as AP due to partial semiological overlap.

### Discussion

The systems and methods herein address the lack of reproducible frameworks for SEEG implantation in temporal lobe epilepsy by introducing a semiology-based approach that links ictal clinical features to functional-anatomical cortical and subcortical regions. Although additional non-invasive data such as structural and functional imaging, neuropsychology, and scalp electrophysiology are routinely incorporated into the construction of implantation geometry and stereotactic coordinates, semiology provides a uniquely direct window into the clinical expression of the seizure network. Because ictal semiology reflects the spatiotemporal dynamics of symptom generation as experienced at the bedside, it serves as a critical first-order anchor for hypothesis generation, onto which other modalities can be layered as refinements. Within this framework, distinct implantation trajectories—mesial/lateral temporal, basal temporal-occipital, anterior perisylvian, and perisylvian—that map onto the functional-anatomical networks most relevant to seizure onset and propagation were identified. By describing these common patterns in anatomical and stereotactic details, and verifying their internal consistency with machine learning, not only a reproducible schema is provided for SEEG electrode implantations but also concrete anatomical guidance for surgical planning. These findings demonstrate that semiology can be systematically translated into implantation strategies that are functionally meaningful and clinically applicable, supporting more precise electrode coverage and, ultimately, more tailored and effective resections. Bridging this gap between bedside clinical manifestations and stereotactic anatomical strategies offers a foundation for greater reproducibility in SEEG planning and for advancing the overall safety and efficacy of epilepsy surgery.

The translation of non-invasive data and localization hypotheses into electrode implantation strategies has long been one of the most challenging steps in presurgical evaluation. Historically, implantation planning has been guided by broad categories, such as “mesial” versus “lateral” temporal lobe epilepsy, which provides a useful clinical shorthand but do not capture the anatomical complexity of seizure networks. This field is advanced by operationalizing these broad categories, including prior descriptions of basal temporal, anterior perisylvian, and perisylvian epilepsies, into well-defined and distinct functional-anatomical schemas with explicit stereotactic correlates. In doing so, it bridges the gap between semiological patterns observed at the bedside and the cortical and subcortical targets that must be sampled to adequately test specific anatomo-electroclinical hypotheses. This functional-anatomical perspective is particularly salient in temporal lobe epilepsy, where seizure propagation frequently extends beyond canonical mesial structures into perisylvian, occipital, or multilobar extra-temporal networks. Accurate characterization of these dynamics necessitates electrode trajectories that extend beyond rigid anatomical boundaries, thereby enabling comprehensive sampling of the epileptogenic network. In this context, the partial overlap observed across implantation patterns should not be regarded as redundancy, but rather as an inherent methodological feature that ensures consistent coverage of critical temporal lobe regions and enhances the robustness of network-based targeting. Importantly, this approach translates into greater clinical applicability, as it provides a reproducible framework for hypothesis-driven implantation that aligns with the complex and distributed nature of seizure networks encountered in surgical practice.

The clinical value of this framework lies in its ability to offer concrete anatomical guidance while remaining flexible enough to adapt to the heterogeneity of patient presentations. By anchoring implantation patterns in reproducible functional-anatomical networks, provided herein is a structured approach that enhances hypothesis generation and facilitates more systematic electrode coverage. In practice, this can improve both the efficiency and safety of SEEG procedures by reducing the likelihood of incomplete sampling or mislocalization. The high seizure freedom rates and favorable neuropsychological outcomes observed in the cohort suggest that such structured approaches, when combined with tailored resections, may optimize both seizure control and preservation of cognitive function compared to standard resections.

Beyond its methodological contributions, this addresses gaps in other approaches: the scarcity of frameworks that translate non-invasive clinical data into anatomical and stereotactic implantation strategies. While imaging modalities such as MRI, PET, and SPECT provide invaluable structural and metabolic information, they cannot capture the dynamic functional anatomy of ictal networks, particularly in non-lesional or discordant cases. Semiology, by contrast, reflects the temporal evolution of seizure propagation in real time and therefore provides a functional entry point into the anatomical dissection of epileptic networks. Here, the results demonstrate that clinically accessible features, such as ictal semiology, can be systematically translated into implantation strategies that are both practical and reproducible. By anchoring stereotactic planning to the clinical expression of seizures, it was shown that implantation patterns can be defined in a manner that remains readily interpretable by clinicians across centers, while still preserving methodological rigor. The internal consistency of these patterns, further validated through computational modeling, underscores their robustness and highlights their potential as a standardized framework for guiding individualized implantation in temporal lobe epilepsy.

The accessibility of this framework should not be understated. Semiology-based analysis requires no advanced technology beyond expert clinical observation, making it a low-cost and widely available tool. For high-resource centers, this framework provides a structured foundation upon which advanced modalities can be layered. For lower-resource centers, particularly those without access to high-cost imaging or specialized ancillary studies, it offers a pragmatic and clinically meaningful approach to SEEG planning. In both contexts, it enhances the reproducibility of electrode implantation strategies, bringing the field closer to standardization without sacrificing the individualized nature of epilepsy surgery.

This analysis showed that semiological features strongly aligned with the real-world electrode coverage decisions (precision 84%, recall 82%). This concordance is not trivial. It demonstrates that clinicians across cases consistently used semiology as a heuristic for implantation, and that this heuristic is reproducible when tested computationally. In other words, the strength of the result lies in showing that subjective semiological interpretation can be formalized, reproduced, and externally verified against independently observed electrode sampling patterns. Thus, the contribution is twofold: 1) validation of clinical reasoning-semiological interpretation, long considered qualitative, can be shown to map consistently onto electrode strategies, reducing variability across centers and 2) framework for harmonization—the reproducibility verified by computational models provides a scalable framework for standardizing implantation approaches and improving comparability of SEEG studies internationally. By reframing semiology as a reproducible driver of implantation-rather than a circular predictor—the analysis highlights its value in harmonizing strategies and informing tailored surgical interventions.

If non-invasive hypothesis classification can predict the location and extent of the epileptogenic zone (EZ) with high accuracy, invasive exploration becomes unnecessary and patients may proceed directly to definitive surgical intervention. This paradigm followed in the practice likely accounts for the relatively low proportion of mesial or lateral temporal hypotheses represented in the cohort (8/60, 13.3%). Patients with mesial temporal lobe epilepsy frequently exhibit highly characteristic semiology in conjunction with concordant imaging and scalp electrophysiological findings, which allows surgical decision-making without the need for SEEG. By contrast, patients referred for intracranial evaluation often present with discordant or ambiguous data, necessitating further clarification through SEEG. As a result, the SEEG cohort described here may not be representative of the broader mesial TLE population. This selection bias may also explain the comparable rate of hippocampal sclerosis observed across the different hypothesis classifications, despite its well-established predominance in mesial temporal lobe epilepsy.

Clinically, this structured approach was associated with a high seizure freedom rate of 75%, comparable or superior to rates reported for standard anterior temporal lobectomy (69%) or selective amygdalohippocampectomy (66%), and potentially favorable relative to laser interstitial thermal therapy (LITT), where seizure freedom ranges from 50-60%. Notably, most resections in the cohort were tailored sublobar procedures rather than standard anterior temporal lobectomies (only 3 of 60 patients underwent standard resections). Neuropsychological outcomes, although preliminary, were encouraging, with verbal memory decline rates after left-sided resections (38%) comparable to or better than prior series reporting rates up to 44-60% for open resections and 35% for LITT. These results emphasize that TLE is not confined to rigid anatomical boundaries but represents a complex functional-anatomical network. In 31% of cases, resections extended beyond the temporal lobe into multilobar regions, including the insula and segments of the frontal, parietal, or occipital lobes. This underscores the need for individualized surgical strategies informed by precise SEEG-guided localization in order to optimize seizure freedom and reduce morbidity.

This was conducted at a single high-volume center with established expertise in orthogonal SEEG implantation. Differences in SEEG philosophy, implantation strategies, including orthogonal vs. oblique implantations, and surgical resources across institutions may limit external validity. While development and validation cohorts were collected at different time points, they reflect a shared institutional philosophy and technical expertise. The role of surgical experience and incremental refinements cannot be excluded, although both cohorts were studied during a mature phase of SEEG practice, minimizing methodological drift. For some subgroups of implantation patterns, numbers are small. This relatively small numbers would indeed be a limitation if the goal were to directly compare outcomes across groups. In this work, however, the primary intention is different: to describe, with unprecedented anatomical detail and stereotactic coordinates, a set of implantation patterns that were derived mainly from non-invasive data-in this case, semiological features.

In this way, the classification serves as a framework for understanding how semiology can inform implantation strategies, rather than as a comparative study of group-level outcomes. These models focused primarily on semiology and anatomical trajectories without incorporating multimodal data such as quantitative imaging, connectivity profiles, or electrophysiological biomarkers, which will be essential for refining predictive accuracy in the future. These limitations, however, do not diminish the central contribution of this work: the demonstration that functional-anatomical frameworks for SEEG implantation can be defined, described, and reproduced from non-invasive clinical data. This characterizes common implantation patterns that emerged over an extended period of surgical practice, with greater consistency and refinement achieved in its later phase, as presented here. These patterns should not be regarded as rigid surgical templates or prescriptive guidelines for electrode placement. Rather, they represent an initial framework of suggested trajectories and anatomical targets that can inform hypothesis-driven sampling, guided by recurrent semiological features, while still allowing for individualized variation according to patient-specific data.

Looking forward, prospective multicenter studies are needed to evaluate reproducibility across diverse patient populations, surgical philosophies, and resource settings. Integration of semiology with modalities such as scalp EEG, MEG, network-level propagation dynamics, resting-state connectivity, and ictal high-frequency oscillations will be an important next step. Just as importantly, implementation into clinical practice will require standardized semiology coding, seamless incorporation into surgical planning software, and clinician training to ensure appropriate use of predictive tools. Together, these efforts can transform semiology from a descriptive clinical art into a structured, anatomically grounded framework that informs surgical decision-making worldwide.

### Conclusion

Presented herein is a reproducible framework for SEEG implantation in TLE that systematically translates ictal semiology into functional-anatomical electrode trajectories. By unifying common implantation schemas, describing them in anatomo-stereotactic details, and verifying their internal consistency, a practical tool that enhances reproducibility and clinical applicability are provided. More broadly, illustrated herein is how non-invasive clinical information can be formalized into concrete anatomical strategies, offering a pathway toward more standardized, precise, and individualized epilepsy surgery.

### Supplementary Methods

### Phase I Evaluation

All patients were referred for Phase I non-invasive evaluation by the treating epileptologist. Phase I evaluation included inpatient scalp video EEG monitoring, high-resolution 3T structural MRI, magnetoencephalography, interictal PET, ictal/interictal SPECT, and neuropsychological evaluation. This information was then reviewed by epileptologists, neurosurgeons, neuroradiologists and neuropsychologists. The hypotheses for the EZ location and its extent, and subsequent decision for intervention (invasive Phase II monitoring, surgical intervention, or continued medical management) were developed by consensus at the conference.

If the patient was referred for Phase II SEEG monitoring, the SEEG trajectory planning was also determined by the conference based on working hypotheses. The working hypotheses guiding the SEEG trajectories took into consideration the conceivable anatomical EZ location and its extent, MRI lesions that were likely related to the organization of seizures, and possible anatomical location of relevant functional cortical areas.

### Neuropsychological Testing

All patients underwent preoperative neuropsychological evaluation by a board eligible or board certified neuropsychologist (L.H., D.C.) including tests of verbal memory (Rey Auditory Verbal Learning Test Delayed Recall-RAVLT DR), visuospatial memory (Rey-Osterrieth Complex Figure Delayed Recall-ROCF DR), picture naming (Boston Naming Test—BNT or Neuropsychological Assessment Battery Naming Test—NAB), phonemic fluency, semantic fluency, and word reading (Wechsler Test of Adult Reading). Patients who subsequently underwent resection/ablation of the EZ were scheduled for 6-month postoperative (7 [6-9] month actual follow-up) neuropsychological testing of similar domains. A decline/improvement was defined as greater than 1 S.D. decrease/increase in the standardized score for each test. Patients with missing neuropsychological follow-up were excluded from analysis of neuropsychological outcomes.

### Preoperative Planning and Surgical Technique

After the working hypotheses were defined, the intended trajectories of electrodes were planned. Using the Talairach stereotaxic space as the primary framework, the trajectories intended to explore the cortical and subcortical structures in a three-dimensional conceptualization, taken into account the distal, intermediate, and proximal cortical subcortical structures traversed by the intracranial electrode. Subsequently, the SEEG intended trajectories were planned on the ROSA system (Zimmer Biomet, Warsaw, IN) using preoperative high-resolution CT, MRI T1 with and without contrast, and CT angiography. Trajectories were planned to maximize gray matter coverage and avoid vascular collisions. Consistent with prior reports, the majority of electrodes were placed orthogonally. Small adjustments (particularly at the entry point) were made to avoid vascular, particularly venous, collisions. Preoperative images were registered and reoriented along the AC-PC line to facilitate planning in Talairach stereotaxic space.

Electrode implantation was performed as previously described. The patients were placed in a Leksell G frame and attached to the ROSA robot. After laser-based registration and verification of registration accuracy, skin incision and skull drilling were performed with a Stryker drill (Strkyer, Kalamazoo, MI) with 2.5 mm bit through the close-fit ROSA adaptor. The metal guiding fixation devices (Dixi Medical, Marchaux-Chaudefontaine, France) were securely screwed into the skull following which the metal stylets were passed to the planned depth. Subsequently, 0.8 mm diameter DIXI electrode with the desired number of electrode contacts is placed. Following the SEEG procedures, high resolution (0.625 mm isotropic resolution) CTs were obtained to assess electrode contact locations and for postoperative hemorrhages. After EMU monitoring phase, electrodes were removed, and the patient was discharged. The decision for subsequent interventions were again made by the multidisciplinary epilepsy patient management conference.

### Electrode Trajectories

All hypothesis classifications usually entailed exploration of the hippocampal formation (anterior and posterior regions), amygdala, entorhinal cortex and temporal pole areas, concomitantly with the exploration of the correspondent lateral and basal temporal cortical areas using orthogonal trajectories. These 5 orthogonal trajectories, which are anatomically described as follows:

Temporal Pole: Interrogation of Brodmann Area 38 is achieved by the implantation of 1 or 2 orthogonally placed electrodes located anterior to the vertical AC line, in the D12 or C10 Talairach coordinate system. Here the trajectories will explore the mesial and lateral aspects of the temporal pole region. Frequently, the C10 trajectory also explore the posterior orbito-frontal cortex (BA 25) using a paralimbic trajectory.

Amygdala: The amygdala is explored by electrodes implanted in orthogonal orientation, immediately posterior to VAC, at the most anterior areas of Talairach coordinate E10. At this coordinate, the trajectory will explore the anterior uncus and the basal lateral nucleus of the amygdaloid complex in the mesial aspect and the dorsal lateral neocortex (BA 21) in the lateral contacts. The amygdala is in proximity with VAC, approximately at 5 mm posterior to the vertical line. The anterior uncus is located at the mesial projection of the amygdaloid nucleus, with the dorsal aspect corresponding to the piriform cortex. In the more lateral aspect of the orthogonal trajectory, the electrode explores the depths of the rostral aspect of the superior temporal sulcus and the crown of the middle temporal gyrus.

Anterior Hippocampus: This is achieved by electrodes implanted in orthogonal orientation, between the VAC and the VPC lines, in the E10 coordinate, but slightly posterior and ventral to the amygdala trajectory. At this coordinate, the trajectory will explore the posterior uncus and the head of the hippocampus in the most mesial aspect and BA 21 located in the rostral aspect of the middle temporal gyrus, in the most lateral aspect.

Posterior Hippocampus: To explore the posterior aspect of the hippocampus body and tail, the orthogonal trajectory is located at the F10 Talairach coordinate. This explores the posterior aspect of the parahippocampal gyrus, the tail of the hippocampus and the depth of the posterior aspect of the superior temporal sulcus, almost at the temporal/parietal/occipital transition, corresponding to the interface between Brodmann areas 22, 21, 39 and 19.

Entorhinal cortex: The entorhinal cortex, with its prominent connections with the head of the hippocampus and the amygdaloid nucleus are important structures to be explored in “dreamer states” scenarios. The same electrode that explores the entorhinal cortex on the mesial contact will cross the depths of the collateral sulcus in the more lateral locations, and subsequent explore the rostral fusiform gyrus, the depth of the temporal-occipital sulcus and will finally explore the inferior temporal gyrus, on tis most lateral trajectory. The trajectory is located immediately ventral to the hippocampus trajectory, at the E11 coordinate.

### Electrode Reconstruction and Areas of Resection

SEEG electrodes reconstructions and coordinate extractions were performed using Curry (Compumedics Neuroscan, Charlotte, North Carolina, USA). Post-SEEG placement high-resolution CTs were registered to preoperative high-resolution MRIs and then to the MNI template. Reconstructions were reviewed by the surgical team to ensure appropriate localization of contacts. The Talairach stereotaxic coordinates of electrode contacts were converted to MNI coordinates and subsequently Brodmann areas using the label4MRI tool which is built on the mni2tal tool in BiolmageSuite and is based off the registration in Lacadie 2009. Brodmann areas were manually inspected for accuracy. For patients who underwent post-SEEG resection, postoperative high resolution T1 MRI was inspected against the Brodmann atlas by the surgical team and authors to determine which Brodmann areas were resected. Resection areas were manually segmented, and volume was calculated in the BrainLab (BrainLab AG, Munich, Germany). Electrode location and trajectory was illustrated over the MNI152 atlas.

### Statistical Analysis and Machine Learning Details

Machine learning was performed using scikit-learn in Python 3.10. Heatmaps and other figures were generated using seaborn. Other statistical analyses were performed in R 4.2.0 (R Foundation for Statistical Computing, Vienna, Austria) with RStudio (Posit Software, Boston, MA). Tables were generated using the gtsummary package. Continuous variables are reported as median [IQR] and categorical as N (%). As appropriate, Fisher's exact test, the Kruskal-Wallis test, and the Chi-squared test were utilized for univariate statistical comparisons. P<0.05 was the prespecified threshold for significance.

### Case Examples

Case 1: Patient is a 56-year-old female with epilepsy since age of 20. Semiology was reported as dizziness followed by left face twitching. In prolonged seizures, the patient was unable to talk and had focal to bilateral secondary generalization. After failing 5 anti-seizure medications, the patient continued to have focal to bilateral tonic-clonic seizures monthly. MRI showed nonspecific white matter changes over the right periventricular regions. EMU phase I data showed right frontocentral onset with rapid secondary synchronization involving bilateral frontocentral regions. SPECT injection (19 seconds) showed increased blood flow over the right insula/perisylvian regions. MEG showed increased irritability in the right posterior perisylvian regions. The proposed hypotheses were right anterior perisylvian vs. lateral temporal vs. frontal. SEEG evaluation showed interictal activity of spikes localized to the lateral STG, synchronous with the mid ventral insula, mid to posterior STG and temporal pole. Ictal EEG was notable for high amplitude spikes over the STG, temporal pole and mesial mid ventral insula followed by low voltage fast activity over the temporal pole followed by posterior STG. Direct cortical stimulation using 50 Hz stimulation of anterior STG triggered the typical stereotypical seizures but not with posterior STG. Patient underwent resection of the temporal pole, anterior STG with sparing of the hippocampus, parahippocampal gyrus, and amygdala. Lateral S involving the anterior superior temporal gyrus with margin of the resection of the mid superior temporal gyrus (T electrode contacts) and resulted in Engel 1D outcome (FIGS. 10A-G).

Case 2: 48-year-old female who presented with epilepsy since her twenties. She describes her seizures as an initial aura of a generalized odd feeling and panic, occasionally associated with a bad odor, progressing to a sense of altered hearing and sensation. The seizure lasts approximately 1 minute and postictally she has headache and fatigue. Her seizures occurred every 1-2 weeks despite failing adequate trials of 6 anti-seizure medications and she had a remote single generalized tonic-clonic seizure. During her phase I video EEG monitoring she was noted to initially look at her left arm and touch it with her right hand and is unable to follow commands or repeat words. She covers her eyes due to reported eye sensitivity and reports continued word finding difficulties postictally. Scalp EEG demonstrates left posterior temporal theta/delta slowing and frequent left posterior temporal spike/polyspikes interictally and left posterior temporal onset ictally. MRI brain demonstrated periventricular nodular heterotopia extending from the left atrium to the left temporal horn as well as atrophy of the perisylvian regions, basal temporal lobe, and cingulate gyrus on the left. Interictal MEG demonstrates left posterior temporal/parietal dipoles. Ictal SPECT (15 second injection) demonstrated increased uptake in the left posterior temporal lobe.

The proposed hypotheses were left perisylvian vs. temporal/basal/occipital vs. mesial temporal. Interictal SEEG evaluation demonstrated near continuous synchronized periodic discharges in the basal temporal and occipital aspects of the heterotopia and cortex but not in other areas, periodic discharges in the cingulate gyrus, and hippocampal head and tail spikes synchronized to discharges from the heterotopia. Ictal EEG demonstrates near synchronous onset from the PVNH and basal temporal/occipital cortices with diffuse fast activity followed by delta slowing with subsequent spread to the more anterior inferior temporal gyrus and posterior cingulate. Stimulation (1 Hz/50 Hz) in basal temporal lobe or cingulate did not elicit any seizures. After multidisciplinary discussion it was felt that both the basal temporal/occipital lobe and the cingulate gyrus could be independent EZs. She was offered resection of the basal temporal/occipital regions with potential second stage ablation of the posterior cingulate gyrus. She underwent the basal temporal/occipital resection (cortical and PVNH) without issue but never required the second stage. She achieved an Engel IA outcome and is weaning antiseizure medications (FIG. 11).

TABLE 1
Cohort characteristics by hypothesis

| | | | | | Temporal/ | | | | | | |
|---|---|---|---|---|---|---|---|---|---|---|---|
| | | | | | basal/ | | Anterior | | | | |
| | Overall, | | Temporal, | | occipital, | | perisylvian, | | Perisylvian, | | |
| Characteristic | N = 60¹ | | N = 8¹ | | N = 10¹ | | N = 20¹ | | N = 22¹ | | p-value² |
| Sex | | | | | | | | | | | 0.3 |
| M | 33 | (55%) | 4 | (50%) | 3 | (30%) | 13 | (65%) | 13 | (59%) | |
| F | 27 | (45%) | 4 | (50%) | 7 | (70%) | 7 | (35%) | 9 | (41%) | |
| Age (years) | 34 | [28, 44] | 34 | [23, 39] | 32 | [28, 48] | 37 | [29, 48] | 33 | [26, 38] | 0.5 |
| MRI abnormality | 40 | (67%) | 3 | (38%) | 8 | (80%) | 14 | (70%) | 15 | (68%) | 0.3 |
| Number of | 14 | [13, 15] | 11 | [9, 13] | 15 | [14, 15] | 14 | [13, 15] | 14 | [14, 16] | 0.044 |
| electrodes | | | | | | | | | | | |
| SEEG laterality | | | | | | | | | 0.4 | | |
| Right | 15 | (25%) | 4 | (50%) | 1 | (10%) | 6 | (30%) | 4 | (18%) | |
| Left | 24 | (40%) | 1 | (13%) | 4 | (40%) | 8 | (40%) | 11 | (50%) | |
| Bilateral | 21 | (35%) | 3 | (38%) | 5 | (50%) | 6 | (30%) | 7 | (32%) | |
| Additional | 1 | (1.7%) | 0 | (0%) | 0 | (0%) | 1 | (5%) | 0 | (0%) | 0.57 |
| electrodes | | | | | | | | | | | |
| required | | | | | | | | | | | |
| Post SEEG | | | | | | | | | 0.9 | | |
| intervention | | | | | | | | | | | |
| None | 4 | (6.7%) | 0 | (0%) | 1 | (10%) | 2 | (10%) | 1 | (4.5%) | |
| Resection | 49 | (82%) | 7 | (88%) | 7 | (70%) | 16 | (80%) | 19 | (86%) | |
| Neuromodulation | 7 | (12%) | 1 | (13%) | 2 | (20%) | 2 | (10%) | 2 | (9.1%) | |
| Extent of | | | | | | | | | 0.3 | | |
| resection³ | | | | | | | | | | | |
| Temporal | 34 | (69%) | 7 | (100%) | 4 | (57%) | 10 | (63%) | 13 | (68%) | |
| Extratemporal ± | 15 | (31%) | 0 | (0%) | 3 | (43%) | 6 | (38%) | 6 | (32%) | |
| temporal | | | | | | | | | | | |
| Resection volume | 30 | [22, 40] | 40 | [29, 41] | 31 | [21, 35] | 28 | [17, 38] | 26 | [22, 32] | 0.4 |
| (cc)³ | | | | | | | | | | | |
| Seizure freedom³ | 36 | (73%) | 6 | (86%) | 5 | (71%) | 12 | (75%) | 13 | (68%) | 0.9 |

¹n (%); Median (IQR)
²Pearson's Chi-squared test; Kruskal-Wallis rank sum test; Fisher's exact test
³Seizure freedom, resection volume computed over resection patients only

SUPPLEMENTARY TABLE 1
Validation cohort details

| | | | Key | | | Resection | |
|---|---|---|---|---|---|---|---|
| | | | Semiological | | Resection | Brodmann | Engel |
| ID | Sex | Classification | Features | GTC | Side | Areas | Outcome |
| S1 | M | Temporal/basal/ | Expressive | N | Left | 38, 27, 28, 34, | 1A |
| | | occipital | speech | | | 35, 36, A, 20, | |
| | | | dysfunction, | | | 37 | |
| | | | visual | | | | |
| | | | autonomic, | | | | |
| | | | deja vu, | | | | |
| | | | anxiety/fear, | | | | |
| | | | behavioral | | | | |
| | | | arrest, | | | | |
| | | | face/oral | | | | |
| | | | automatisms, | | | | |
| S2 | M | Anterior | Autonomic, | Y | Right | 38, 27, 28, 34, | 2B |
| | | perisylvian | anxiety/fear, | | | 35, 36, A, | |
| | | | behavioral | | | HH, 20 | |
| | | | arrest, gen. | | | | |
| | | | body | | | | |
| | | | sensation, | | | | |
| | | | face/oral | | | | |
| | | | automatisms, | | | | |
| | | | contralateral | | | | |
| | | | eye | | | | |
| | | | deviation, | | | | |
| | | | hand | | | | |
| | | | automatisms, | | | | |
| | | | hand/arm/leg | | | | |
| | | | motor | | | | |
| S3 | M | Anterior | Autonomic, | N | Right | 38, 27, 28, 34, | 1A |
| | | perisylvian | behavioral | | | 35, 36, A, | |
| | | | arrest, | | | HH, HT, 20 | |
| | | | face/oral | | | | |
| | | | automatisms, | | | | |
| | | | contralateral | | | | |
| | | | eye | | | | |
| | | | deviation, | | | | |
| | | | versive head | | | | |
| | | | movements, | | | | |
| | | | hand | | | | |
| | | | automatisms, | | | | |
| | | | hand/arm/leg | | | | |
| | | | motor | | | | |
| S4 | M | Anterior | Autonomic, | Y | Left | 38, 27, 28, 34, | 1A |
| | | perisylvian | deja vu, | | | 35, 36, A, | |
| | | | behavioral | | | HH, 20, 11 | |
| | | | arrest, | | | | |
| | | | face/oral | | | | |
| | | | automatisms, | | | | |
| | | | facial | | | | |
| | | | expression, | | | | |
| | | | hand | | | | |
| | | | automatisms, | | | | |
| | | | hand/arm/leg | | | | |
| | | | motor, visual | | | | |
| S5 | F | Anterior | Behavioral | N | Left | 38, 27, 28, 34, | 1D |
| | | perisylvian | arrest, | | | 35, 36, A | |
| | | | face/oral | | | | |
| | | | automatisms, | | | | |
| | | | other facial | | | | |
| | | | movement, | | | | |
| | | | expressive | | | | |
| | | | speech, | | | | |
| | | | contralateral | | | | |
| | | | eye | | | | |
| | | | deviation, | | | | |
| | | | hand | | | | |
| | | | automatisms, | | | | |
| | | | hand/arm/leg | | | | |
| | | | motor | | | | |
| S6 | F | Anterior | Behavioral | Y | Left | 38, 27, 28, 34, | 1A |
| | | perisylvian | arrest, | | | 35, 36, A, 20 | |
| | | | face/oral | | | | |
| | | | automatisms, | | | | |
| | | | expressive | | | | |
| | | | speech, | | | | |
| | | | contralateral | | | | |
| | | | eye | | | | |
| | | | deviation, | | | | |
| | | | versive head, | | | | |
| | | | hand | | | | |
| | | | automatisms | | | | |
| S7 | F | Perisylvian | Other facial | Y | Left | 22, 21 | 2B |
| | | | movement, | | | | |
| | | | contralateral | | | | |
| | | | eye | | | | |
| | | | deviation, | | | | |
| | | | Versive | | | | |
| | | | head, | | | | |
| | | | Auditory | | | | |
| S8 | M | Perisylvian | Behavioral | Y | Left | 38, 27, 28, 34, | 1A |
| | | | arrest, | | | 35, 36, A, | |
| | | | Face/oral | | | HH, 20 | |
| | | | automatisms, | | | | |
| | | | contralateral | | | | |
| | | | eye | | | | |
| | | | deviation, | | | | |
| | | | versive head, | | | | |
| | | | hand | | | | |
| | | | automatisms, | | | | |
| | | | hand/arm/leg | | | | |
| | | | motor, visual | | | | |
| S9 | M | Perisylvian | Autonomic, | Y | Left | 38, 27, 28, 34, | 1A |
| | | | behavioral | | | 35, 36, A, | |
| | | | arrest, gen. | | | HH, 20 | |
| | | | body | | | | |
| | | | sensation, | | | | |
| | | | face/oral | | | | |
| | | | Automatisms | | | | |
| S10 | M | Perisylvian | Behavioral | Y | Right | 40 | 1A |
| | | | arrest, gen. | | | | |
| | | | body | | | | |
| | | | sensation, | | | | |
| | | | face/oral | | | | |
| | | | automatisms, | | | | |
| | | | other facial | | | | |
| | | | movement, | | | | |
| | | | hand | | | | |
| | | | automatisms, | | | | |
| | | | hand/arm/leg | | | | |
| | | | motor | | | | |
| S11 | M | Perisylvian | Behavioral | Y | Right | 38, 27, 28, 34, | 1A |
| | | | arrest, gen. | | | 35, 36, A | |
| | | | body | | | | |
| | | | sensation, | | | | |
| | | | face/oral | | | | |
| | | | automatisms, | | | | |
| | | | facial | | | | |
| | | | expression | | | | |
| S12 | M | Perisylvian | Behavioral | Y | Left | 37, 18, 19 | 1A |
| | | | arrest, other | | | | |
| | | | facial | | | | |
| | | | movement, | | | | |
| | | | contralateral | | | | |
| | | | eye | | | | |
| | | | deviation, | | | | |
| | | | hand/arm/leg | | | | |
| | | | motor, visual | | | | |

SUPPLEMENTARY TABLE 2
Electrode targeting coordinates and nomenclature

| | | Indirect targeting coordinates [median, | | | | | |
|---|---|---|---|---|---|---|---|
| | | (IQR)] | | | | | |
| Electrode | | Talairach | | | MNI | | |
| Abbreviation | Anatomic Target | X | Y | Z | X | Y | Z |
| A | Amygdala | ±15.7 | −3.7 | −12.4 | ±17 | −4 | −17 |
| | | (3.5) | (5.4) | (7.4) | (3) | (5) | (8) |
| B | Head of | ±20.1 | −14.3 | −13.4 | ±21 | −15 | −17 |
| | hippocampus | (4.5) | (5.7) | (5.6) | (4) | (7) | (6) |
| C | Tail of | ±14.5 | −32.2 | −4.6 | ±15 | −33 | −7 |
| | Hippocampus | (4.8) | (6) | (8.1) | (4) | (7) | (8) |
| E | Entorhinal cortex | ±21.9 | −9.3 | −24.4 | ±22 | −11 | −30 |
| | | (4.9) | (6.5) | (5.4) | (6) | (8) | (7) |
| F | Fusiform gyrus | ±30.3 | −45.5 | −17.5 | ±31 | −48 | −20 |
| | | (6.4) | (7) | (8.2) | (9) | (7) | (10) |
| Fr | Frontal pole | ±3.2 | 53.5 | 12.8 | ±4 | 59 | 7 |
| | | (3.1) | (4.9) | (11.2) | (4) | (4) | (13) |
| I | Temporal pole | ±23.5 | 8.5 | −26.5 | ±25 | 8 | −34 |
| | | (5.6) | (6.9) | (5.6) | (5) | (7) | (7) |
| J | Orbitofrontal cortex - | ±4.3 | 12.6 | −8.8 | ±5 | 13 | −14 |
| | anterior STG | (4.6) | (3.9) | (6.6) | (6) | (4) | (8) |
| | (paralimbic | | | | | | |
| | electrodes) | | | | | | |
| O | Lingual gyrus - | ±16.1 | −65.6 | −12.5 | ±16 | −69 | −14 |
| | occipital lobe | (14.8) | (17.7) | (13.7) | (13) | (19) | (16) |
| Or | Orbitofrontal cortex - | ±3.3 | 37.1 | −2.85 | ±3 | 39 | −9 |
| | pars orbitalis | (7.8) | (8.4) | (8.4) | (10) | (10) | (10) |
| Q | Pars triangularis / | ±28.3 | 12.3 | 10.6 | ±30 | 15 | 7 |
| | operculum - | (6.9) | (8.1) | (5.1) | (8) | (8) | (6) |
| | anterior | | | | | | |
| | insula | | | | | | |
| R | Inferior precentral | ±32.3 | −6.0 | 12.2 | ±33 | −5 | 10 |
| | gyrus - mid insula | (3.2) | (8.1) | (5.2) | (5) | (8) | (6) |
| S | Inferior postcentral | ±31.9 | −15.6 | 14.1 | ±34 | −15 | 14 |
| | gyrus - posterior | (5.9) | (7.6) | (8.2) | (5) | (8) | (9) |
| | insula | | | | | | |
| T | Planum polare - | ±34.6 | −2.7 | −1 | ±36 | −2 | −5 |
| | anterior insula | (4.6) | (6.4) | (6.1) | (4) | (7) | (7) |
| U | Planum temporale - | ±29.5 | −21.2 | 5.6 | ±31 | −21 | 4 |
| | Heschl's gyrus - | (5.2) | (9.7) | (5.2) | (5) | (10) | (6) |
| | posterior insula | | | | | | |
| V | Cuneus - occipital | ±3.4 | −73.1 | 10.6 | ±4 | −75 | 13 |
| | lobe | (2.8) | (8) | (13.6) | (4) | (8) | (16) |
| Z | Anterior cingulate - | ±4.1 | 35.2 | 21.2 | ±5 | 39 | 18 |
| | frontal lobe | (7.8) | (4.8) | (8) | (8) | (5) | (9) |
| X | Posterior cingulate - | ±3.0 | −48.0 | 18.5 | ±3 | −49 | 20 |
| | supramarginal | (3.3) | (8.7) | (10.5) | (3) | (10) | (11) |
| | gyrus | | | | | | |

SUPPLEMENTARY TABLE 3
Key features by hypotheses per recursive feature elimination analysis.

| | | | Temporal | | | |
|---|---|---|---|---|---|---|
| | | | Basal | Anterior | | Feature |
| | | Temporal | Occipital | perisylvian | Perisylvian | Importance |
| Semiology | Behavioral | 10.00% | 16.70% | 26.70% | 46.70% | 0.132 |
| | arrest | | | | | |
| | Autonomic | 15.40% | 11.50% | 23.10% | 50.00% | 0.125 |
| | Visual | 22.20% | 55.60% | 0.00% | 22.20% | 0.123 |
| | Face/oral | 13.30% | 13.30% | 46.70% | 26.70% | 0.112 |
| | automatisms | | | | | |
| | Generalized | 7.70% | 30.80% | 46.20% | 15.40% | 0.101 |
| | body | | | | | |
| | sensation | | | | | |
| | Expressive | 10.00% | 30.00% | 30.00% | 30.00% | 0.092 |
| | speech | | | | | |
| | Deja vu | 33.30% | 33.30% | 22.20% | 11.10% | 0.086 |
| | Versive | 10.00% | 10.00% | 40.00% | 40.00% | 0.08 |
| | head | | | | | |
| | Anxiety/fear | 0.00% | 28.60% | 57.10% | 14.30% | 0.076 |
| | Dreamy | 50.00% | 0.00% | 25.00% | 25.00% | 0.074 |
| | state | | | | | |
| Electrode | Prefrontal | 0.00% | 0.00% | 80.00% | 31.80% | 0.128 |
| Location | 10 | | | | | |
| | M1 4 | 12.50% | 10.00% | 75.00% | 63.60% | 0.121 |
| | Parietal 40 | 25.00% | 50.00% | 10.00% | 68.20% | 0.116 |
| | Cingulate | 50.00% | 80.00% | 10.00% | 68.20% | 0.108 |
| | 23 | | | | | |
| | IFG 44 | 50.00% | 0.00% | 85.00% | 54.50% | 0.105 |
| | Occipital 18 | 25.00% | 90.00% | 0.00% | 36.40% | 0.093 |
| | Entorhinal | 100.00% | 80.00% | 75.00% | 50.00% | 0.087 |
| | 28 | | | | | |
| | Hippo. Tail | 87.50% | 100.00% | 65.00% | 68.20% | 0.084 |
| | Premotor 6 | 37.50% | 30.00% | 90.00% | 81.80% | 0.082 |
| | Orbitofrontal | 25.00% | 0.00% | 85.00% | 40.90% | 0.078 |
| | 11 | | | | | |

SUPPLEMENTARY TABLE 4
Hypothesis classification performance using Bernoulli Naïve Bayes classifier
of semiological (a = 0.001) and electrode location (Brodmann areas) (a = 0.01) features

| s | Cohort | Classification | Precision | Recall | FI-Score | Support |
|---|---|---|---|---|---|---|
| Semiology | Main/Train | Temporal | 0.600 | 0.375 | 0.462 | 8 |
| | (N = 60) | TBO | 0.857 | 0.600 | 0.706 | 10 |
| | | Ant. | 0.640 | 0.800 | 0.711 | 20 |
| | | perisylvian | | | | |
| | | Perisylvian | 0.609 | 0.636 | 0.622 | 22 |
| | | Macro | 0.676 | 0.603 | 0.625 | 60 |
| | | Average | | | | |
| | | Weighted | 0.659 | 0.650 | 0.644 | 60 |
| | | Average | | | | |
| | | Overall | 0.650 | | | |
| | | Classifier | | | | |
| | | Accuracy | | | | |
| | Validation | Temporal | — | — | — | 0 |
| | (N = 12) | TBO | 1.000 | 1.000 | 1.000 | 1 |
| | | Ant. | 0.500 | 0.600 | 0.545 | 5 |
| | | perisylvian | | | | |
| | | Perisylvian | 0.750 | 0.500 | 0.600 | 6 |
| | | Macro | 0.563 | 0.525 | 0.536 | 12 |
| | | Average | | | | |
| | | Weighted | 0.667 | 0.583 | 0.611 | 12 |
| | | Average | | | | |
| | | Overall | 0.583 | | | |
| | | Classifier | | | | |
| | | Accuracy | | | | |
| Electrode | Main/Train | Temporal | 0.636 | 0.875 | 0.737 | 8 |
| Location | (N = 60) | TBO | 0.833 | 1.000 | 0.909 | 10 |
| | | Ant. | 0.895 | 0.850 | 0.872 | 20 |
| | | Perisylvian | | | | |
| | | Perisylvian | 0.944 | 0.773 | 0.850 | 22 |
| | | Macro | Average | 0.827 | 0.874 | 0.842 |
| | | Weighted | 0.868 | 0.850 | 0.852 | 60 |
| | | Average | | | | |
| | | Overall | 0.850 | | | |
| | | Classifier | | | | |
| | | Accuracy | | | | |
| | Validation | Temporal | — | — | — | 0 |
| | (N = 12) | TBO | 0 | 0 | 0 | 1 |
| | | Ant. | 1.000 | 0.600 | 0.750 | 5 |
| | | Perisylvian | | | | |
| | | Perisylvian | 0.714 | 0.833 | 0.769 | 6 |
| | | Macro | 0.429 | 0.358 | 0.380 | 12 |
| | | Weighted | 0.774 | 0.667 | 0.697 | 12 |
| | | Average | | | | |
| | | Overall | 0.667 | | | |
| | | Classifier | | | | |
| | | Accuracy | | | | |

SUPPLEMENTARY TABLE 5
Random forests classifier (Number of estimators: 200, no maximum
depth, minimum samples per split: 10, minimum samples per
leaf: 1) mapping seizure semiology to electrode coverage

| | | | | F1 | F1 |
|---|---|---|---|---|---|
| | | Precision | Recall | Micro | Macro |
| | Overall | 0.841 | 0.824 | 0.832 | 0.660 |
| Train | Temporal | 0.924 | 0.918 | 0.921 | 0.619 |
| (N = 60) | TBO | 0.869 | 0.817 | 0.842 | 0.526 |
| | Ant. Perisylvian | 0.847 | 0.827 | 0.847 | 0.651 |
| | Perisylvian | 0.796 | 0.794 | 0.795 | 0.622 |
| Validation | Overall | 0.647 | 0.711 | 0.677 | 0.458 |
| (N = 12) | Temporal | — | — | — | — |
| | TBO | 0.684 | 0.591 | 0.634 | 0.317 |
| | Ant. Perisylvian | 0.647 | 0.824 | 0.725 | 0.401 |
| | Perisylvian | 0.641 | 0.651 | 0.646 | 0.438 |

SUPPLEMENTARY TABLE 6
Key resection features by hypotheses per recursive feature elimination analysis.

| | | | Temporal | | | |
|---|---|---|---|---|---|---|
| | | | Basal | Anterior | | Feature |
| | | Temporal | Occipital | perisylvian | Perisylvian | Importance |
| Resection | Temporal | 100.00% | 71.40% | 62.50% | 52.60% | 0.153 |
| | Pole 38 | | | | | |
| | Hippocampal | 14.30% | 0.00% | 37.50% | 26.30% | 0.123 |
| | Tail | | | | | |
| | Orbitofrontal | 0.00% | 0.00% | 31.20% | 5.30% | 0.12 |
| | 11 | | | | | |
| | ITG 20 | 14.30% | 57.10% | 0.00% | 26.30% | 0.116 |
| | Amygdala | 100.00% | 57.10% | 56.20% | 63.20% | 0.107 |
| | Occipital 19 | 0.00% | 42.90% | 0.00% | 15.80% | 0.094 |
| | IFG 47 | 0.00% | 0.00% | 31.20% | 5.30% | 0.09 |
| | Perirhinal 35 | 71.40% | 57.10% | 50.00% | 52.60% | 0.079 |
| | Fusiform 37 | 0.00% | 42.90% | 0.00% | 21.10% | 0.063 |
| | MTG 21 | 0.00% | 14.30% | 6.20% | 21.10% | 0.055 |

SUPPLEMENTARY TABLE 7
Hypothesis classification performance using Bernoulli Naïve Bayes
classifier of resection location (Brodmann areas) features (a = 0.8)

| | Cohort | Classification | Precision | Recall | FI-Score | Support |
|---|---|---|---|---|---|---|
| Resection | Main/Train | Temporal | 0.000 | 0.000 | 0.000 | 7 |
| | (N = 49) | TBO | 0.000 | 0.000 | 0.000 | 7 |
| | | Ant. | 0.833 | 0.313 | 0.455 | 16 |
| | | perisylvian | | | | |
| | | Perisylvian | 0.419 | 0.947 | 0.581 | 19 |
| | | Macro | 0.313 | 0.315 | 0.259 | 49 |
| | | Average | | | | |
| | | Weighted | 0.434 | 0.469 | 0.374 | 49 |
| | | Average | | | | |
| | | Overall | 0.469 | | | |
| | | Classifier | | | | |
| | | Accuracy | | | | |
| | Validation | Temporal | — | — | — | 0 |
| | (N = 12) | TBO | 0.000 | 0.000 | 0.000 | 1 |
| | | Ant. | 1.000 | 0.200 | 0.333 | 5 |
| | | perisylvian | | | | |
| | | Perisylvian | 0.545 | 1.000 | 0.706 | 6 |
| | | Macro | 0.515 | 0.400 | 0.346 | 0.515 |
| | | Average | | | | |
| | | Weighted | 0.689 | 0.583 | 0.492 | 0.689 |
| | | Average | | | | |
| | | Overall | 0.583 | | | |
| | | Classifier | | | | |
| | | Accuracy | | | | |

SUPPLEMENTARY TABLE 8
Factors associated with seizure freedom in
all subjects with post-SEEG intervention

| | Not Seizure Free, | Seizure Free, | |
|---|---|---|---|
| Characteristic | N = 20¹ | N = 36¹ | p-value² |
| Sex | | | 0.5 |
| M | 10 (50%) | 21 (58%) | |
| F | 10 (50%) | 15 (42%) | |
| Age | 33 (29, 44) | 34 (26, 44) | >0.9 |
| MRI Abnormality | 12 (60%) | 25 (69%) | 0.5 |
| Number of Electrodes | 15 (13, 15) | 14 (13, 15) | 0.8 |
| Laterality | | | 0.4 |
| Right | 3 (15%) | 11 (31%) | |
| Left | 9 (45%) | 14 (39%) | |
| Bilateral | 8 (40%) | 11 (31%) | |
| Post SEEG intervention | | | <0.001 |
| Resection | 13 (65%) | 36 (100%) | |
| Neuromodulation | 7 (35%) | 0 (0%) | |
| Extent of resection | | | 0.2 |
| Temporal | 7 (54%) | 27 (75%) | |
| Extratemporal + temporal | 6 (46%) | 9 (25%) | |

¹n (%); Median (IQR)
²Fisher's exact test; Pearson's Chi-squared test; Wilcoxon rank sum test

SUPPLEMENTARY TABLE 9
Neuropsychological outcomes in resection/ablation
cohort (including validation cohort)

| Neuropsychological Test | Overall | Left | Right | p-value¹ |
|---|---|---|---|---|
| Verbal memory | | | | 0.14 |
| (RAVLT DR) | | | | |
| Decline | 9 (26%) | 8 (38%) | 1 (7.7%) | |
| Stable | 20 (59%) | 10 (48%) | 10 (77%) | |
| Improved | 5 (15%) | 3 (14%) | 2 (15%) | |
| Visuospatial memory | | | | 0.3 |
| (ROCF DR) | | | | |
| Decline | 2 (6.5%) | 2 (9.5%) | 0 (0%) | |
| Stable | 21 (68%) | 15 (71%) | 6 (60%) | |
| Improved | 8 (26%) | 4 (19%) | 4 (40%) | |
| Picture Naming | | | | 0.019 |
| (BNT/NAB) | | | | |
| Decline | 9 (28%) | 9 (41%) | 0 (0%) | |
| Stable 1 | 9 (59%) | 12 (55%) | 7 (70%) | |
| Improved | 4 (13%) | 1 (4.5%) | 3 (30%) | |
| Phonemic Fluency | | | | 0.63 |
| Decline | 3 (10%) | 3 (15%) | 0 (0%) | |
| Stable | 25 (83%) | 17 (85%) | 8 (80%) | |
| Improved | 2 (6.7%) | 0 (0%) | 2 (20%) | |
| Semantic Fluency | | | | 0.046 |
| Decline | 11 (37%) | 9 (45%) | 2 (20%) | |
| Stable | 14 (47%) | 10 (50%) | 4 (40%) | |
| Improved | 5 (17%) | 1 (5.0%) | 4 (40%) | |
| Word reading (WTAR) | | | | 0.2 |
| Decline | 2 (7.1%) | 2 (12%) | 0 (0%) | |
| Stable | 25 (89%) | 15 (88%) | 10 (91%) | |
| Improved | 1 (3.6%) | 0 (0%) | 1 (9.1%) | |

### B. Systems and Methods for Identifying Locations to Place Stereo-Electroencephalography (SEEG) Electrodes on Brain Tissues of Subjects for Detection of Epileptogenic Zones (EZs)

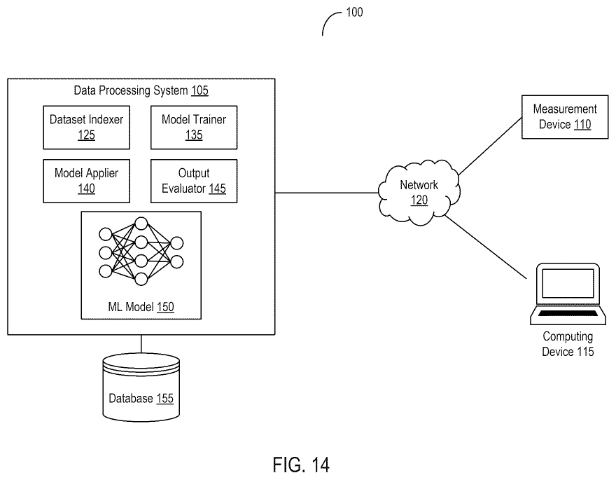

Referring to FIG. 14, depicted is a block diagram of a system 100 for identifying locations to place stereo-electroencephalography (SEEG) electrodes on brain tissues of subjects for detection of epileptogenic zones (EZs). In overview, the system 100 may include at least one data processing system 105, at least one measurement device 110, at least one computing device 115, and at least one database 155, among others, communicatively coupled with one another via at least one network 120. The data processing system 105 may include at least one data indexer 125, at least one model trainer 135, at least one model applier 140, at least one output evaluator 145, and at least one machine learning (ML) model 150, among others. Each of the components in the system 100 (such as the data processing system 105 and its subcomponents and the computing device 115) as detailed herein may be implemented using hardware (e.g., one or more processors coupled with memory), or a combination of hardware and software as detailed herein in Section B. The system 100 may be used to implement the functionalities described herein in Section A.

In further detail, the data processing system 105 may be any computing device including one or more processors coupled with memory and software and capable of performing the various processes and tasks described herein. The data processing system 105 may be associated with an entity to process neuroimages and data associated with subjects at risk for or diagnosed with epilepsy to provide output. The data processing system 105 can be in communication with the measurement device 110, the computing device 115, the database 155, and other devices, via the network 120. The data processing system 105 may be situated, located, or otherwise associated with at least one server group. The server group may correspond to a data center, a branch office, or a site at which one or more servers corresponding to the data processing system 105 is situated.

The data processing system 105 may include one or more modules, components, or subsystems to perform the various processes and tasks described herein. The dataset indexer 125 may receive datasets acquired for a subject from the measurement device 110. The model trainer 135 may initialize, train, and establish the ML model 150. The model applier 140 may apply a sample to the ML model, generate an output, and store the output. The output evaluator 145 may use the ML model 150 to determine locations to place stereo-electroencephalography (SEEG) electrodes on brain tissues of subjects for detection of epileptogenic zones (EZs).

The ML model 150 may include any type of machine learning (ML) or artificial intelligence (AI) architecture to process subject datasets. The architecture for the ML model 150 may include, for example, a deep learning artificial neural network (ANN) (e.g., an encoder of a convolutional neural network (CNN), a Markov chain, a support vector machine (SVM), a clustering algorithm, a Bayesian classifier, or a decision tree, among others. In general, the ML model 150 may include at least one input, at least one output, and a set of weights relating the input with the output. The input may include a dataset for a given subject, which may include at least a neuroimage and a metric of the subject. The output may include coordinates relating to the locations to place the SEEG electrodes on the subject. The set of weights may be arranged in accordance with the ML or AI architecture.

The measurement device 110 may be an instrument or device to collect a dataset from a subject. For example, the measurement device 110 may collect at least one of a neuroimage or a metric corresponding to the subject. The measurement device 110 may collect the neuroimage in accordance with at least one of a plurality of modalities. For example, the measurement device 110 may collect the neuroimage with at least one of the following modalities: a position emission tomography (PET), magnetic resonance imaging (MRI), electroencephalography (EEG), single-photon emission computed tomography (SPECT), or magnetoencephalography (MEG), among others.

In some embodiments, the measurement device 110 may also collect a metric corresponding to the subject. The metric may indicate the degree of neuropsychological function in at least one of a plurality of neuropsychological evaluations. For example, the metric may correspond with at least one of the following neuropsychological evaluations: a verbal test, a visuospatial memory test, a picture naming test, a phonemic fluency test, a semantic fluency test, or a word reading test, among others. The measurement device 110 may be configured to collect additional subject data. For example, the measurement device 110 may collect at least one of a plurality of traits, a clinical history, an epilepsy history, or a plurality of semiology characteristics of the subject, among others.

The computing device 115 (sometimes herein referred to as an end user computing device) may be any computing device comprising one or more processors coupled with memory and software and capable of performing the various processes and tasks described herein. The computing device 115 may be in communication with the data processing system 105, the measurement device 110, and the database 155 via the network 120. The computing device 115 may have at least one display. The computing device 115 may be associated with an entity (e.g., a clinician) examining a subject at risk for or diagnosed with epilepsy. The display may present information about the subject provided by the data processing system 105.

The database 155 may store and maintain various resources and data associated with the data processing system 105, the measurement device 110, and the computing device 115, among others. The database 155 may include a database management system (DBMS) to arrange and organize the data maintained thereon. The database 155 may be in communication with the data processing system 105, the measurement device 110, and the computing device 115, via the network 120. While running various operations, the data processing system 105, the measurement device 110, and the computing device 115 may access the database 155 to retrieve identified data therefrom. The data processing system 105, the measurement device 110, and the computing device 115 may also write data onto the database 155 from running such operations.

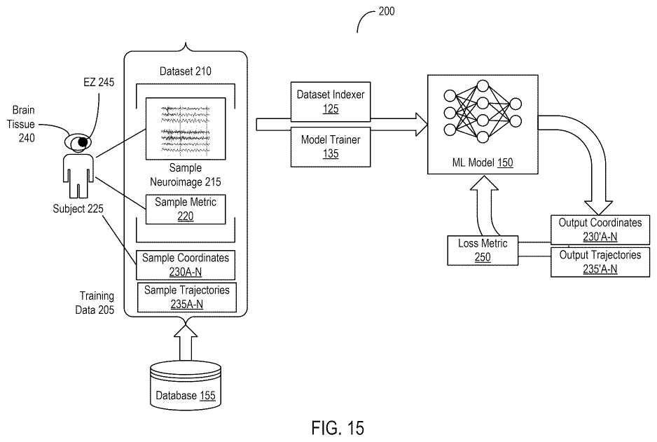

Referring now to FIG. 15, depicted is a block diagram of a process 200 of training a machine learning (ML) model to identify locations to place SEEG electrodes on brain tissues of subjects for detection of EZs. The process 200 may include or correspond to operations performed in the system 100 to establish the ML model 150. Under the process 200, the dataset indexer 125 executing on the data processing system 105 may retrieve, obtain, or otherwise identify the training data 205. In some embodiments, the dataset indexer 125 may access the database 155 to retrieve the training data 205. The training data 205 may be used to train the ML model 150. The training data 205 may include a set of examples. Each example may include at least one dataset 210 (sometimes herein referred to as a semiology data). The dataset 210 may include at least one sample neuroimage 215 and at least one sample metric 220 for a sample subject 225. The subject 225 may be a human or animal subject, among others. The subject 225 may have, may be diagnosed with or may be at risk of, epilepsy. The epilepsy may include focal epilepsy, such as temporal lobe epilepsy (TLE), frontal lobe epilepsy, parietal lobe epilepsy, occipital lobe epilepsy, among others. In some embodiments, the epilepsy may include generalized epilepsy, such as absence seizures, myoclonic seizures, clonic seizures, tonic seizures, tonic-clonic seizures, or atonic seizures, among others.

The sample neuroimage 215 may be a neuroimage or an electrophysiological recording of at least one brain of the subject 225. The neuroimage 215 may be in accordance with at least one of the following modalities: positron emission tomography (PET), magnetic resonance imaging (MRI), electroencephalography (EEG), single-photon emission computed tomography (SPECT), or magnetoencephalography (MEG), among others. The sample neuroimage 215 may contain a variety of subject data relating to the epilepsy within the subject 225. For example, the neuroimage 215 may provide data relating to anatomy, electrical activity, metabolic activity, or blood flow of the brain of the subject 225, among others.

The sample metric 220 may be a metric indicating a degree of a respective neuropsychological function of the subject 225. The sample metric may be obtained via at least one of the following neuropsychological evaluations: a verbal test, a visuospatial memory test, a picture naming test, a phonemic fluency test, a semantic fluency test, or a word reading test, among others. In some embodiments, the output of the at least one neuropsychological evaluation may be a numeric value that indicates a level of neuropsychological function of the subject 225. The value of neuropsychological function may include a value relating to attention, memory, language, executive function, or visuospatial skills, among others.

The training data 205 may include additional subject data. For example, the dataset 210 may include at least one of a plurality of traits, a clinical history, an epilepsy history, or a plurality of semiology characteristics of the subject 225, among others. The plurality of traits may correspond to demographic data relating to the subject 225. For example, the plurality of traits may correspond to demographic traits of the subject such as age, gender, or background of the subject 225, among others. The training data 205 may also include a clinical history of the subject 225. For example, the clinical history may include family medical history, medication history, known allergies, and previous treatments of the subject 225, among others. The training data 205 may include an epilepsy history of the subject 225, which may include age of onset of epilepsy, description of seizures, seizure triggers, and so on. The training data 205 may include a plurality of semiology characteristics of the subject 225. The plurality of semiology characteristics may correspond to specific symptoms the seizures of the subject 225. For example, the plurality of semiology characteristics may include pre-seizure symptoms, post-seizure symptoms, types of movements during seizures, duration of seizures, and emotional responses to seizures, among others. The additional subject data may be used to further train the ML model 150.

The training dataset may include sample coordinates 230A-N (hereinafter generally referred to as sample coordinates 230) and sample trajectories 235A-N (hereinafter generally referred to as sample trajectories 235). The sample coordinates 230 may identify a plurality of locations at which to place a plurality of SEEG electrodes on the brain tissue 240 to detect an EZ 245 within the brain of the subject 225. In some embodiments, the sample coordinates may be presented according to a coordinate system, such as Montreal Neurological Institute (MNI) coordinates or Talairach coordinates, among others. The sample trajectories 235 may identify at least one of an angle or a depth of placement for the SEEG electrodes on the brain tissue 240 to detect the EZ 245 within the brain tissue 240 of the subject 225. Each of the sample trajectories 235 may define the orientation the SEEG electrodes in the brain tissue 240 of the subject 225 to detect the EZ 245. For example, the brain tissue 240 of the subject 225 may correspond at least one of an amygdala, a head of hippocampus, a tail of hippocampus, an entorhinal cortex, a fusiform gyrus, a frontal pole, a temporal pole, an orbitofrontal cortex, a lingual gyrus, a pars triangularis, an inferior precentral gyrus, a planum temporale, a cuneus, an anterior cingulate, or a posterior cingulate, among others.

In some embodiments, the training data 205 may identify or include a respective classification for each sample coordinate 230 (or sample trajectory 235). The classification may define an anatomical region in the brain tissue 240 on which the respective SEEG electrode at the sample coordinate 230 is to be placed. The classification may include, for example, mesial/lateral temporal (MLT) region, temporal/basal/occipital (TBO) region, anterior perisylvian (AP) region, or perisylvian (P) region, among others. The MLT region may correspond to inner (mesial) and outer (lateral) portions of the temporal lobe of the brain tissue 240. The TBO region may correspond to inferior temporal cortex, fusiform gyrus, parts of the basal occipito-temporal cortex, or ventral occipital region of the brain tissue 240. The AP region may correspond to a front portion of the sylvian fissure region separating the frontal and temporal lobes of the brain tissue 240. The P region may correspond to a region surrounding the Sylvian fissure of the brain tissue 240.

Using the training data 205, the model trainer 135 may initialize, establish, and train the ML model 150. In some embodiments, the model trainer 135 may update one or more parameters of the ML model 150. In some embodiments, the model trainer 135 may generate at least one tree in the ML model 150 comprising a random forest using the training data 205 and the sample coordinates 230 to train the ML model 150. For instance, with a random forest as the ML model 150, the model trainer 135 may randomly sample (e.g., via bootstrapping or bagging) examples of the training data 205 to create multiple decision trees for the random forest. At each node, the model trainer 135 may determine whether to split the decision tree further via feature selection to de-correlate the decision tree in the random forest of the ML model 150. The model trainer 135 may perform splitting recursively to separate target variables. The model trainer 135 may then combine the decision trees to perform ensemble learning. With the completion of ensemble learning, the model trainer 135 may use the remaining set of examples in the training data 205 to evaluate the accuracy of prediction and update the weights of the ML model 150.

In some embodiments, the model trainer 135 may use supervised learning techniques to update the ML model 150 (e.g., deep neural network). For example, the model trainer 135 may apply the dataset 210 of the training data 205 to the ML model 150. Using the dataset 210, the ML model 150 may generate an output using patterns from the training data 205. The model trainer 135 may then compare the output to one or more parameters of the training data 205 to generate a loss metric 250. Using the loss metric 250, the model trainer 135 may then update one or more plurality of parameters of the ML model 150.

The model trainer 135 executing on the data processing system 105 may input, feed, or otherwise apply the training data 205 to the ML model 150 to generate output coordinates 230′A-N (hereinafter generally referred to as output coordinates 230′) and output trajectories 235′A-N (hereinafter generally referred to as output trajectories 235′). The output coordinates 230′ may generally be a plurality of locations to place a plurality of SEEG electrodes along a brain tissue of a second subject. The output trajectories 235′ may generally be a plurality of trajectories for the corresponding plurality of locations (e.g., output coordinates 230′). The output trajectories 235′ may define at least one of an angle or depth of placement for the SEEG electrodes along the brain tissue of the second subject. Upon training of the ML model 150, the output coordinates 230′ and the output trajectories 235′ have similar to or equal values as the sample coordinates 230 and the sample trajectories 235′.

In conjunction, the model trainer 135 may calculate, generate, or otherwise determine at least one loss metric 250 based on a comparison between the sample coordinates 230A-N and the sample trajectories 235A-N from the training data 205 and the output coordinates 230′A-N and output trajectories 235′A-N from the ML model 150. The error metric may indicate a degree of deviation of the output (e.g., output coordinates 230′ and output trajectories 235′) from the expected result (e.g., sample coordinates 230 and sample trajectories 235). The error metric in accordance with any number of loss functions, such as a norm loss (e.g., L1 or L2), mean absolute error (MAE), mean squared error (MSE), a quadratic loss, a cross-entropy loss, and a Huber loss, among others.

Using the loss metric 250, the model trainer 135 may modify, change, or otherwise update at least one parameter of the ML model 150. The updating may be in accordance with a backpropagation algorithm and an objective function. The objective function may define one or more rates at which the weights are to be updated. The objective function may be in accordance with stochastic gradient descent, and may include, for example, an adaptive moment estimation (Adam), implicit update (ISGD), and adaptive gradient algorithm (AdaGrad), among others. The updating of the weights of the ML model 150 may be repeated until convergence.

The model trainer 135 may store the updated parameters of the ML model 150 in the database 155. For example, the model trainer 135 may store and maintain the plurality of parameters of the ML model 150 to be used to generate the output coordinates 230′ and the output coordinates 230′ for a plurality of locations to place a plurality of SEEG electrodes along a brain tissue of a second subject. The model trainer 135 may store the plurality of parameters using one or more data structures. The data structures may include, for example, an array, a matrix, a linked list, a stack, a tree, a hash table, or an object, among others.

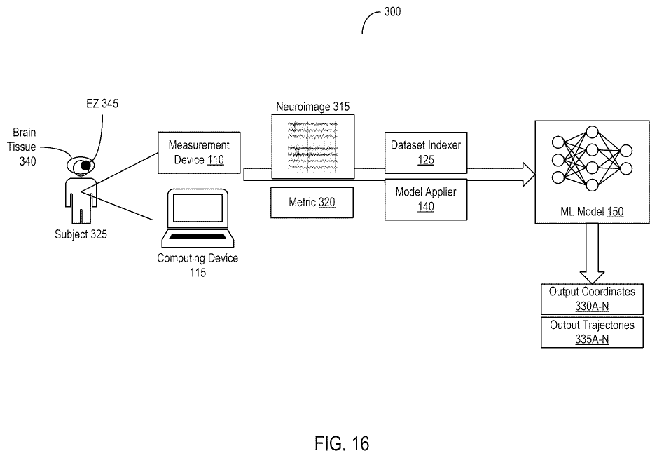

Referring now to FIG. 16, depicted is a block diagram of a process 300 for applying a model to identify locations to place SEEG electrodes on brain tissues of subjects for detection of EZs. The process 300 may include or correspond to operations performed in the system 100 to apply the ML model 150 to generate output coordinates from subject data. Under the process 300, the dataset indexer 125 on the data processing system 105 may retrieve, identify, or otherwise receive at least one neuroimage 315 and at least one metric 320 for at least one subject 325. The subject 325 may be a human or an animal subject, among others. The subject 325 may have, may be diagnosed with or may be at risk of, epilepsy. The epilepsy may include, for example, focal epilepsy (e.g., temporal lobe epilepsy (TLE), frontal lobe epilepsy, parietal lobe epilepsy, occipital lobe epilepsy) or generalized epilepsy (e.g., absence seizures, myoclonic seizures, clonic seizures, tonic seizures, tonic-clonic seizures, or atonic seizures), among others.

The subject 325 may have at least one brain, comprising at least one brain tissue 340 at which SEEG electrodes may be placed. The brain tissue 340 may correspond to at least one of a plurality of anatomical sites. For example, the brain tissue 340 of the subject 325 may be brain tissue of at least one of an amygdala, a head of hippocampus, a tail of hippocampus, an entorhinal cortex, a fusiform gyrus, a frontal pole, a temporal pole, an orbitofrontal cortex, a lingual gyrus, a pars triangularis, an inferior precentral gyrus, a planum temporale, a cuneus, an anterior cingulate, or a posterior cingulate, among others. The brain of the subject 325 may include at least one EZ 345. The EZ 345 may be at an unknown location within the brain of the subject 325.

In some embodiments, the dataset indexer 125 may obtain, fetch, or otherwise acquire the neuroimage 315 via the measurement device 110. The measurement device 110 may acquire, scan, or otherwise generate the neuroimage 315 of at least one brain of the subject 325. The neuroimage 315 may contain or include raw data acquired by the measurement device in accordance with an imaging modality (e.g., positron emission tomography (PET), magnetic resonance imaging (MRI), electroencephalography (EEG), single-photon emission computed tomography (SPECT), magnetoencephalography (MEG), etc.). The neuroimage 315 may be a neuroimage of at least one brain of the subject 325. The neuroimage 315 may contain a variety of subject data relating to the epilepsy within the subject 325. For example, the neuroimage 315 may provide data relating to anatomy, electrical activity, metabolic activity, or blood flow of the brain of the subject 325, among others. Upon acquisition, the measurement device 110 may send, transmit, or otherwise provide the neuroimage 315 to the data processing system 105.

In some embodiments, the dataset indexer 125 may obtain, fetch, or otherwise acquire the metric 320 via the computing device 115. The metric 320 may indicate the degree of neuropsychological function in accordance with at least one of a plurality of neuropsychological evaluations. For example, the plurality of neuropsychological evaluations may include at least one of a verbal test, a visuospatial memory test, a picture naming test, a phonemic fluency test, a semantic fluency test, or a word reading test, among others. In some embodiments, the output of the at least one neuropsychological evaluation may be a numeric value that indicates a level of neuropsychological function of the subject 325. The value of neuropsychological function may include a value relating to attention, memory, language, executive function, or visuospatial skills, among others. Upon acquisition, the computing device 115 may send, transmit, or otherwise provide the metric 320 to the data processing system 105.

With the receipt, the model applier 140 may input, feed, or otherwise apply the neuroimage 315 and the metric 320 to the ML model 150. In some embodiments, the model applier 140 may carry out or perform a transformation on the input data (e.g., the neuroimage 315 and the metric 320). For example, the model applier 140 may transform the data to a domain readable by the ML model 150, such as the frequency domain. The transformation may include, for example, a Fourier transform, a Wavelet transform, discrete cosine transform (DCT), or a Hilbert transform, among others. The model applier 140 may apply the transformed data from the neuroimage 315 and the metric 320 to the ML model 150. The model applier 140 may process the neuroimage 315 and the metric 320 in accordance with the weights of the ML model 150.

The model applier 140 executing on the data processing system 105 may create, produce, or otherwise generate at least the output coordinates 330A-N (hereinafter generally referred to as output coordinates 330) and the output trajectories 335A-N (hereinafter generally referred to as output trajectories 335). The output coordinates 330 and the output trajectories 335 may be in accordance with a set coordinate system, such as Talairach and MNI coordinates. The output coordinates 330 may refer to a plurality of coordinates for a corresponding plurality of locations along the brain tissue 340 of the subject 325. Each of the plurality of output coordinates 330 identify a corresponding location of the plurality of locations at which to place the SEEG electrodes on the brain tissue 340 to detect an EZ 345 within the brain of the subject 325. The output trajectories 335 may refer to a plurality of trajectories for the corresponding plurality of locations of the output coordinates 330. Each trajectory of the plurality of output trajectories 335 may define at least one of an angle or a depth of placement for the SEEG electrode of the plurality of SEEG electrodes. The output trajectories 335 may provide improved accuracy of placement of the SEEG electrodes on or near the EZ 345.

In some embodiments, the model applier 140 may determine, from a set of classifications, a respective classification for each coordinate 330 (or each trajectory 335) based on applying the ML model 150. The classification may define an anatomical region in the brain tissue 240 on which the respective SEEG electrode at the sample coordinate 230 is to be placed. The classification may include, for example, mesial/lateral temporal (MLT) region, temporal/basal/occipital (TBO) region, anterior perisylvian (AP) region, or perisylvian (P) region, among others. The MLT region may correspond to inner (mesial) and outer (lateral) portions of the temporal lobe of the brain tissue 240. The TBO region may correspond to inferior temporal cortex, fusiform gyrus, parts of the basal occipito-temporal cortex, or ventral occipital region of the brain tissue 240. The AP region may correspond to a front portion of the sylvian fissure region separating the frontal and temporal lobes of the brain tissue 240. The P region may correspond to a region surrounding the Sylvian fissure of the brain tissue 240. The plurality of classifications may be used to classify the plurality of coordinates 330 and the plurality of trajectories 335 for the subject 325.

The model applier 140 may store and maintain an association between the subject 325 (e.g., using a subject identifier) and at least one of the output coordinates 330 and/or the output trajectories 335 on the database 155 using one or more data structures. The association may be a link, a map, or a relation between the subject 325 and at least one of the output coordinates and/or the output trajectories 335. The one or more data structures can include an array, a linked list, a stack, a tree, or a hash table, among others. The association may be also with at least one of the neuroimage 315 or the metric 320 and information related to the subject 325, such as parameters characterizing at least one of the neuroimage 315 or the metric 320 at least in partial concurrence to the subject 325. For example, the association may include any abnormalities seen on the neuroimage 315 corresponding to the subject 325.

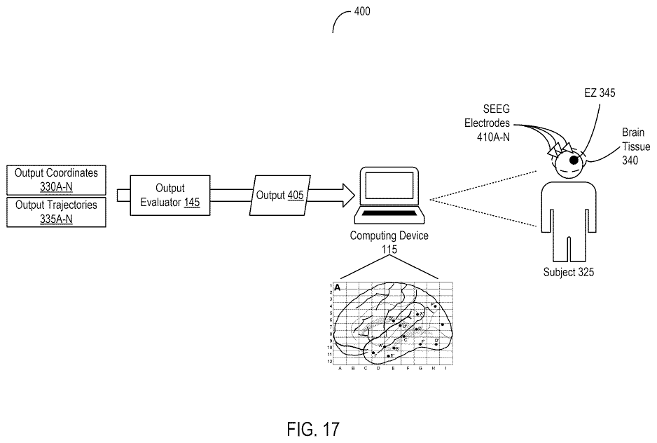

Referring now to FIG. 17, a block diagram of a process 400 to produce outputs in the system for identifying locations to place SEEG electrodes. The process 400 may include or correspond to operations performed in the system 100 to produce the outputs for identifying locations to place SEEG electrodes. Under the process 400, the output evaluator 145 executing on the data processing system 105 may create, produce, or otherwise generate at least one output 405. In some embodiments, the output evaluator 145 may transmit the output 405 to the computing device 115. The output 405 may be used to place SEEG electrodes on a subject for detection of an EZ.

Under the process, the output evaluator 145 may create, produce, or otherwise generate at least one output 405 based on the output coordinates 330 and the output trajectories 335. The output 405 may include information associated with the output coordinates 330 and the output trajectories 335. For example, the output 405 may include a set of instructions for using the output coordinates 330 and the output trajectories 335 to place the SEEG electrodes to identify the EZ 345. In some embodiments, where the model applier 140 created a classification for the output coordinates 330 and the output trajectories 335, the output 405 may include information associated with the classification. For example, when the classification indicates that the output coordinates 330 and output trajectories are consistent with the MLT classification, the output 405 may include the indication of the MLT classification.

In some embodiments, the output 405 may identify or include a plurality of locations at which to place the plurality of SEEG electrodes 410A-N (hereinafter generally referred to as SEEG electrodes 410) on the brain tissue 340 of the subject 325. The output 405 may be generated based on the output coordinates 330A-N and the output trajectories 335. In some embodiments, the output 405 may include at least one of the set of coordinates 330 or the set of trajectories 335. The plurality of locations may correspond to the ideal locations to place the SEEG electrodes 410 to detect the EZ 345 in the subject 325.

In some embodiments, the output evaluator 145 may identify or determine the type of epilepsy in the subject 325 based on the classifications for the set of coordinates 330 (or the trajectories 335). The type of epilepsy may include, for example, focal epilepsy (e.g., temporal lobe epilepsy (TLE), frontal lobe epilepsy, parietal lobe epilepsy, occipital lobe epilepsy) or generalized epilepsy (e.g., absence seizures, myoclonic seizures, clonic seizures, tonic seizures, tonic-clonic seizures, or atonic seizures), among others. The output evaluator 145 may use a mapping between the classifications for the set of coordinates 330 and the type of epilepsy. For instance, the mapping may specify, if the classifications for the set of coordinates 330 correspond to temporal lobe regions, the output evaluator 145 may determine that that type of epilepsy in the subject 325 is TLE. The output evaluator 145 may include an identification of the type of epilepsy in the output 405.

With the generation, the output evaluator 145 may send, transmit, or otherwise provide the output 405 to the computing device 115. With receipt, the computing device 115 may display, render, or otherwise present the output 405 including the information on the classification for the subject 325. The information of the output 405 may be presented via a graphical user interface on a display of the computing device 115. The information of the output 405 may be used to make clinical decisions. For example, the information of the output 405 may include instructions described herein for placing electrodes to detect the EZ 345. The information of the output 405 may also be used for additional clinical decisions, such as plan of treatment for the subject 325.

According to the output 405, the SEEG electrodes 410A-N may be placed (e.g., by a surgeon) in the brain tissue 340 of the subject 325 to detect the EZ 345. The SEEG electrodes 410 may be inserted, arranged, or otherwise placed at the output coordinates 330 on the brain tissue 340. The orientation and depth of the SEEG electrodes 410 may also be situated, inserted, or otherwise arranged according to the output trajectories 335. The output 405 may identify an anatomical site of the brain tissue 340 at which to place the SEEG electrodes 410A-N (hereinafter generally referred to as SEEG electrodes 410). For example, the output 405 may identify instructions to place the SEEG electrodes 410 on or near an amygdala, a head of hippocampus, a tail of hippocampus, an entorhinal cortex, a fusiform gyrus, a frontal pole, a temporal pole, an orbitofrontal cortex, a lingual gyrus, a pars triangularis, an inferior precentral gyrus, an inferior postcentral gyrus, a planum temporale, a cuneus, an anterior cingulate, or a posterior cingulate, among others. The display of the output 405 may be used to guide the placement of the SEEG electrodes 410. For example, the display may include a mapping of the brain of the subject 325 with an overlay of the desired location of the SEEG electrodes 410. The mapping may be followed during the placement of the SEEG electrodes 410.

With the placement of the SEEG electrodes 410 on the brain tissue 340, SEEG data corresponding to brain activity used to detect the EZ 345 in the brain tissue 340 in the brain of the subject 325 may be measured via the SEEG electrodes 410. The SEEG electrodes 410 may be configured to detect a plurality of electrical impulses generated within the brain tissue 340 as a result of brain activity. In this regard, the SEEG electrodes 410 may be able to detect the EZ 345 due to the increased brain activity associated with seizures. The SEEG data measured by the SEEG electrodes 410 may be collected and transmitted to the computing system and stored, for example in the database 155. The SEEG data may include electrophysiological signals (e.g., time-series data) measuring brain activity within the brain of the subject 325. Using the electrophysiological signals, the EZ 345 may be detected within the brain of the subject 325. The SEEG data may be collected over an appropriate period, such as multiple days. Collection of SEEG data from the SEEG electrodes 410 over a long period of time ensures that the SEEG electrodes 410 detected periods of normal brain activity and brain activity relating to a seizure of the subject 325.

In this manner, by leveraging the ML model 150, the data processing system 105 may generate output coordinates 330 and output trajectories 335 using multimodal data (e.g., a neuroimage 315 and a metric 320 of a subject 325). The output coordinates 330 and output trajectories 335 correspond to a predicted location of an EZ 345 within the subject 325, which allows for the SEEG electrodes 410 to be strategically placed within the brain of the subject 325 for identification of the EZ 345. These outputs may provide performance advantages relative to other approaches to placement SEEG electrodes 410.

From a clinical perspective, relative to other approaches (e.g., those that do not rely on machine learning techniques as described herein), the likelihood of accurately and precisely detecting the EZ may be greatly improved. Furthermore, the output 405 including the set of coordinates 330 and trajectories 335 may be individually tailored, allowing clinicians to implement placement strategies specific to a particular subject 325, rather than relying on broad anatomical templates. This may allow for better clinical outcomes, including improved seizure control and preservation of cognitive function, when the output 405 is used to guide surgery. In other techniques, the SEEG electrodes 410 may be incorrectly positioned and unable to detect the EZ 345 at the correct location. Alternatively, the clinician may have to manually and repeatedly place the SEEG electrodes 410 in an attempt to detect and localize the EZ 345. This is undesirable from a clinical standpoint, as the process of collecting SEEG data is both time and resource consuming.

From a computer resource perspective, by using the ML model 150, the data processing system 105 may allow for more efficient use of computing resources (e.g., processing, memory, and network bandwidth) that otherwise would have been wasted in providing inaccurate or useless output from processing subject data (e.g., neuroimages and metrics). In addition, the data processing system 105 may use the ML model 150 on the multimodal data to extract complex features that would have otherwise not been inferred from the data in a single modality or by manual evaluation by a clinician. The presentation of the output 405 via the computing device 110 may enhance the quality of human-machine interface (HMI) between the user and the computing device 110.

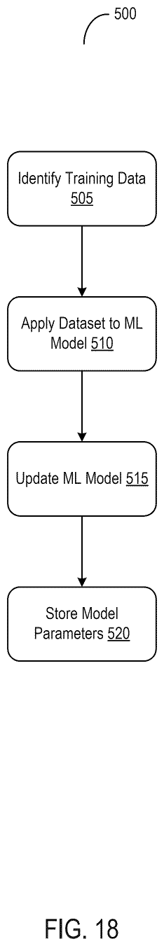

Referring now to FIG. 18, depicted is a flow diagram of a method 500 of training a machine learning (ML) model. The method 500 may be implemented by any components detailed herein, such as the system 100 or 700. Under the method 500, a computing system may identify a training dataset (505). The computing system may apply the training dataset to a machine learning (ML) model (510). The computing system may update the ML model using the output generated by the dataset (515). The computing system may store the updated model parameters in a database (520).

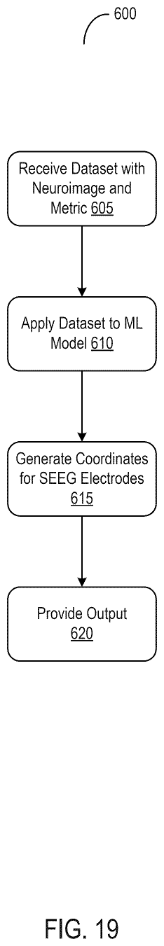

Referring now to FIG. 19, depicted is a flow diagram of a method 600 of identifying locations to place SEEGs. The method 600 may be implemented by any components detailed herein, such as the system 100 or 700. Under the method 600, a computing system may receive a dataset with a neuroimage and a metric (605). The neuroimage and the metric may correspond to a subject who is at risk of, or has been diagnosed with, epilepsy. The computing system may then apply the dataset to a machine learning (ML) model (610). As an output of the ML model, the computing system may generate coordinates for SEEG electrodes (615). The coordinates may indicate a plurality of coordinates within the brain at which to place a plurality of electrodes in order to detect an epileptogenic zone (EZ). The computing system may provide an output based on the generated coordinates (620). The output may identify a corresponding plurality of locations at which to place the plurality of SEEGs on the brain tissue of the subject.

### C. Network and Computing Environment

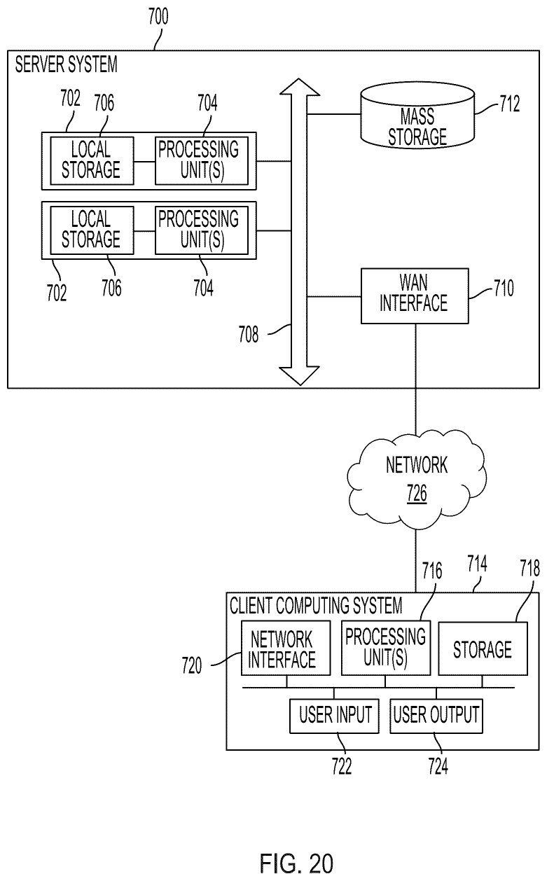

Various operations described herein can be implemented on computer systems. FIG. 20 shows a simplified block diagram of a representative server system 700, computing system 714, and network 726 usable to implement certain embodiments of the present disclosure. In various embodiments, server system 700 or similar systems can implement services or servers described herein or portions thereof. Computing system 714 or similar systems can implement clients described herein. The system 100 described herein can be similar to the server system 700. Server system 700 can have a modular design that incorporates a number of modules 702 (e.g., blades in a blade server embodiment); while two modules 702 are shown, any number can be provided. Each module 702 can include processing unit(s) 704 and local storage 706.

Processing unit(s) 704 can include a single processor, which can have one or more cores, or multiple processors. In some embodiments, processing unit(s) 704 can include a general-purpose primary processor as well as one or more special-purpose co-processors such as graphics processors, digital signal processors, or the like. In some embodiments, some, or all processing units 704 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 704 can execute instructions stored in local storage 706. Any type of processors in any combination can be included in processing unit(s) 704.

Local storage 706 can include volatile storage media (e.g., DRAM, SRAM, SDRAM, or the like) and/or non-volatile storage media (e.g., magnetic, or optical disk, flash memory, or the like). Storage media incorporated in local storage 706 can be fixed, removable, or upgradeable as desired. Local storage 706 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 704 need at runtime. The ROM can store static data and instructions that are needed by processing unit(s) 704. The permanent storage device can be a non-volatile read-and-write memory device that can store instructions and data even when module 702 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 706 can store one or more software programs to be executed by processing unit(s) 704, such as an operating system and/or programs implementing various server functions such as functions of the system 100 or any other system described herein, or any other server(s) associated with system 100 or any other system described herein.

“Software” refers generally to sequences of instructions that, when executed by processing unit(s) 704, cause server system 700 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 704. Software can be implemented as a single program or a collection of separate programs or program modules that interact as desired. From local storage 706 (or non-local storage described below), processing unit(s) 604 can retrieve program instructions to execute and data to process in order to execute various operations described above.

In some server systems 700, multiple modules 702 can be interconnected via a bus or other interconnect 708, forming a local area network that supports communication between modules 702 and other components of server system 700. Interconnect 708 can be implemented using various technologies, including server racks, hubs, routers, etc.

A wide area network (WAN) interface 710 can provide data communication capability between the local area network (e.g., through the interconnect 708) and the network 726, such as the Internet. Other technologies can be used to communicatively couple the server system 700 with the network 726, including wired (e.g., Ethernet, IEEE 802.3 standards) and/or wireless technologies (e.g., Wi-Fi, IEEE 802.11 standards).

In some embodiments, local storage 706 is intended to provide working memory for processing unit(s) 704, providing fast access to programs and/or data to be processed while reducing traffic on interconnect 708. Storage for larger quantities of data can be provided on the local area network by one or more mass storage 712 that can be connected to interconnect 708. Mass storage 712 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage 712. In some embodiments, additional data storage resources may be accessible via WAN interface 710 (potentially with increased latency).

Server system 700 can operate in response to requests received via WAN interface 710. For example, one of modules 702 can implement a supervisory function and assign discrete tasks to other modules 702 in response to received requests. Work allocation techniques can be used. As requests are processed, results can be returned to the requester via WAN interface 710. Such operation can generally be automated. Further, in some embodiments, WAN interface 710 can connect multiple server systems 700 to each other, providing scalable systems capable of managing high volumes of activity. Other techniques for managing server systems and server farms (collections of server systems that cooperate) can be used, including dynamic resource allocation and reallocation.

Server system 700 can interact with various user-owned or user-operated devices via a wide-area network such as the Internet. An example of a user-operated device is shown in FIG. 20 as computing system 714. Computing system 714 can be implemented, for example, as a consumer device such as a smartphone, other mobile phone, tablet computer, wearable computing device (e.g., smart watch, eyeglasses), desktop computer, laptop computer, and so on.

For example, computing system 714 can communicate via WAN interface 710. Computing system 714 can include computer components such as processing unit(s) 716, storage device 718, network interface 720, user input 722, and user output 724. Computing system 714 can be a computing device implemented in a variety of form factors, such as a desktop computer, laptop computer, tablet computer, smartphone, other mobile computing device, wearable computing device, or the like.

Processing unit 716 and storage device 718 can be similar to processing unit(s) 704 and local storage 706 described above. Suitable devices can be selected based on the demands to be placed on computing system 714. For example, computing system 714 can be implemented as a “thin” client with limited processing capability or as a high-powered computing device. Computing system 714 can be provisioned with program code executable by processing unit(s) 716 to enable various interactions with server system 700.

Network interface 720 can provide a connection to the network 726, such as a wide area network (e.g., the Internet) to which WAN interface 710 of server system 700 is also connected. In various embodiments, network interface 720 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 722 can include any device (or devices) via which a user can provide signals to computing system 714; computing system 714 can interpret the signals as indicative of particular user requests or information. In various embodiments, user input device 722 can include any or all of a keyboard, touch pad, touch screen, mouse, or other pointing device, scroll wheel, click wheel, dial, button, switch, keypad, microphone, and so on.

User output device 724 can include any device via which computing system 714 can provide information to a user. For example, user output device 724 can include display-to-display images generated by or delivered to computing system 714. The display can incorporate various image generation technologies, e.g., a liquid crystal display (LCD), light-emitting diode (LED) display including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 724 can be provided in addition to or instead of a display. Examples include indicator lights, speakers, tactile “display” devices, printers, and so on.

Some embodiments include electronic components, such as microprocessors, storage, and memory that store computer program instructions in a computer readable storage medium. Many of the features described in this specification can be implemented as processes that are specified as a set of program instructions encoded on a computer readable storage medium. When one or more processing units execute these program instructions, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 704 and 716 can provide various functionality for server system 700 and computing system 714, including any of the functionality described herein as being performed by a server or client, or other functionality.

It will be appreciated that server system 700 and client computing system 714 are illustrative and that variations and modifications are possible. Computer systems used in connection with embodiments of the present disclosure can have other capabilities not specifically described here. Further, while server system 700 and client computing system 714 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of identifying locations to place stereo-electroencephalography (SEEG) electrodes on brain tissues of subjects for detection of epileptogenic zones (EZs), comprising:
receiving, by one or more processors, for a subject at risk of or diagnosed with epilepsy, a dataset comprising a neuroimage of a brain of the subject;
applying, by the one or more processors, the neuroimage of the dataset to a machine learning (ML) model;
generating, by the one or more processors, based on applying the dataset to the ML model, a plurality of coordinates for a corresponding plurality of locations along a brain tissue of the subject, each of the plurality of coordinates identifying a corresponding location of the plurality of locations at which to place an SEEG electrode of a plurality of SEEG electrodes on the brain tissue to detect an EZ within the brain of the subject; and
storing, by the one or more processors, using one or more data structures, an association between the subject and the plurality of coordinates.

2. The method of claim 1, further comprising providing, by the one or more processors, for presentation via a user interface, an output based on the plurality of coordinates identifying the corresponding plurality of locations at which to place the plurality of SEEG electrodes on the brain tissue of the subject.

3. The method of claim 1, wherein receiving the dataset further comprises receiving the dataset comprising the neuroimage in accordance with at least one of a plurality of modalities, the plurality of modalities comprising a positron emission tomography (PET), magnetic resonance imaging (MRI), electroencephalography (EEG), single-photon emission computed tomography (SPECT), or magnetoencephalography (MEG).

4. The method of claim 1, wherein receiving the dataset further comprises receiving the dataset comprising at least one of:
(i) a metric indicating a degree of neuropsychological function in accordance with at least one of a plurality of neuropsychological evaluations, the plurality of neuropsychological evaluations comprising a verbal test, a visuospatial memory test, a picture naming test, a phonemic fluency test, a semantic fluency test, or a word reading test,
(ii) a plurality of traits,
(iii) a clinical history,
(iv) an epilepsy history, or
(v) a plurality of semiology characteristics of the subject.

5. The method of claim 1, wherein generating the plurality of coordinates further comprises generating a plurality of trajectories for the corresponding plurality of locations, each trajectory of the plurality of trajectories defining at least one of an angle or a depth of placement for the SEEG electrode of the plurality of SEEG electrodes.

6. The method of claim 1, wherein generating the plurality of coordinates further comprises determining, for each of the plurality of coordinates, a classification of a plurality of classifications,
wherein the plurality of classifications comprises at least one of mesial/lateral temporal (MLT) region, temporal/basal/occipital (TBO) region, anterior perisylvian (AP) region, or perisylvian (P) region.

7. The method of claim 1, wherein SEEG data corresponding to brain activity used to detect the EZ in the brain is measured via the plurality of SEEG electrodes placed along the brain tissue at the plurality of locations identified by the plurality of coordinates.

8. The method of claim 1, wherein an anatomical site of the brain tissue to be placed with the plurality of SEEG electrodes corresponds to at least one of an amygdala, a head of hippocampus, a tail of hippocampus, an entorhinal cortex, a fusiform gyrus, a frontal pole, a temporal pole, an orbitofrontal cortex, a lingual gyrus, an orbitofrontal cortex, a pars triangularis, an inferior precentral gyrus, an inferior postcentral gyrus, a planum temporale, a cuneus, an anterior cingulate, or a posterior cingulate.

9. A method of training models to identify locations to place stereo-electroencephalography (SEEG) electrodes on brain tissues of subjects for detection of epileptogenic zones (EZs), comprising:
identifying, by one or more processors, an example from a plurality of examples, each example of the plurality of examples for a first subject at risk of or diagnosed with epilepsy, comprising:
(i) a first dataset including a neuroimage of a first brain of the first subject, and
(ii) a first plurality of coordinates for a first plurality of locations along a first brain tissue of the first subject, each of the first plurality of coordinates identifying a respective location of the first plurality of locations at which to place a corresponding SEEG electrode of a first plurality of SEEG nodes on the brain tissue to detect an EZ within the brain;
applying, by the one or more processors, at least the first dataset of the example to a machine learning (ML) model comprising a plurality of parameters;
updating, by the one or more processors, one or more of the plurality of parameters of the ML model, based on applying at least the dataset of the example to the ML model; and
storing, by the one or more processors, the plurality of parameters of the ML model to be used to generate a second plurality of coordinates for a second plurality of locations to place a second plurality of SEEG electrodes along a brain tissue of a second subject.

10. The method of claim 9, wherein identifying the example further comprises selecting the example from a second plurality of examples different from the plurality of examples,
wherein updating one or more of the plurality of parameters of the ML model further comprises generating at least one tree in the ML model comprising a random forest using the first dataset and the first plurality of coordinates, in response to applying the example to the ML model.

11. The method of claim 9, wherein applying at least the first dataset of the example to the ML model further comprises applying the first dataset of the example to the ML model to generate a second plurality of coordinates for a third plurality of locations to place a second plurality of SEEG electrodes along the brain tissue of the first subject, and
wherein updating the one or more of the plurality of parameters further comprises updating the one or more of the plurality of parameters based on a comparison between the first plurality of coordinates and the second plurality of coordinates.

12. The method of claim 9, wherein the neuroimage is in accordance with at least one of a plurality of modalities, the plurality of modalities comprising a positron emission tomography (PET), magnetic resonance imaging (MRI), electroencephalography (EEG), single-photon emission computed tomography (SPECT), or magnetoencephalography (MEG).

13. The method of claim 9, wherein the first dataset comprises at least one of:
(i) a metric indicating a degree of neuropsychological function in accordance with at least one of a plurality of neuropsychological evaluations, the plurality of neuropsychological evaluations comprising a verbal test, a visuospatial memory test, a picture naming test, a phonemic fluency test, a semantic fluency test, or a word reading test,
(ii) a plurality of traits,
(iii) a clinical history,
(iv) an epilepsy history, or
(v) a plurality of semiology characteristics of the subject.

14. The method of claim 9, wherein the first dataset comprises, for each of the first plurality of coordinates, a respective classification of a plurality of classifications, wherein the plurality of classifications comprises at least one of mesial/lateral temporal (MLT) region, temporal/basal/occipital (TBO) region, anterior perisylvian (AP) region, or perisylvian (P) region.

15. A system for identifying locations to place stereo-electroencephalography (SEEG) electrodes on brain tissues of subjects for detection of epileptogenic zones (EZs), comprising:
one or more processors coupled with memory, configured to:
receive, for a subject at risk of or diagnosed with epilepsy, a dataset comprising a neuroimage of a brain of the subject;
apply the neuroimage of the dataset to a machine learning (ML) model;
generate, based on applying the dataset to the ML model, a plurality of coordinates for a corresponding plurality of locations along a brain tissue of the subject, each of the plurality of coordinates identifying a corresponding location of the plurality of locations at which to place an SEEG electrode of a plurality of SEEG electrodes on the brain tissue to detect an EZ within the brain of the subject; and
store, using one or more data structures, an association between the subject and the plurality of coordinates.

16. The system of claim 15, wherein the one or more processors are further configured to provide, for presentation via a user interface, an output based on the plurality of coordinates identifying the corresponding plurality of locations at which to place the plurality of SEEG electrodes on the brain tissue of the subject.

17. The system of claim 15, wherein the one or more processors are further configured to receive the dataset comprising the neuroimage in accordance with at least one of a plurality of modalities, the plurality of modalities comprising a positron emission tomography (PET), magnetic resonance imaging (MRI), electroencephalography (EEG), single-photon emission computed tomography (SPECT), or magnetoencephalography (MEG).

18. The system of claim 15, wherein the one or more processors are further configured to receive the dataset comprising a metric indicating a degree of neuropsychological function in accordance with at least one of a plurality of neuropsychological evaluations, the plurality of neuropsychological evaluations comprising a verbal test, a visuospatial memory test, a picture naming test, a phonemic fluency test, a semantic fluency test, or a word reading test.

19. The system of claim 15, wherein the one or more processors are further configured to generate a plurality of trajectories for the corresponding plurality of locations, each trajectory of the plurality of trajectories defining at least one of an angle or a depth of placement for the SEEG electrode of the plurality of SEEG electrodes.

20. The system of claim 15, wherein SEEG data corresponding to brain activity used to detect the EZ in the brain is measured via the plurality of SEEG electrodes placed along the brain tissue at the plurality of locations identified by the plurality of coordinates.
